(19) United States
(12) Patent Application Publication
(10) Pub. No.: US 2009/0239223 A1
(43) Pub. Date: Sep. 24, 2009

(54) Prediction of Breast Cancer Response to Taxane-Based Chemotherapy

(75) Inventors: Mathias Gehrmann, Leverkusen (DE); Christian Von Törne, Solingen (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS HEALTHCARE DIAGNOSTICS INC., Tarrytown, NY (US)

(21) Appl. No.: 12/307,590

(22) PCT Filed: Jul. 6, 2007
(86) PCT No.: PCT/EP07/05998

§ 371 Date: Jan. 6, 2009

(30) Foreign Application Priority Data
Jul. 13, 2006 (EP) 06014557.0

(51) Int. Cl. C12Q 1/68

(52) U.S. Cl. 435/6

(57) ABSTRACT

The invention relates to methods and kits for the prediction of a likely outcome of chemotherapy in a cancer patient. More specifically, the invention relates to the prediction of tumour response to chemotherapy based on measurements of expression levels of a small set of marker genes. The set of marker genes is useful for the identification of breast cancer subtypes responsive to taxane based chemotherapy, such as e.g. a taxane-anthracycline-cyclophosphamide-based (e.g. Taxotere (docetaxel)-Adriamycin (doxorubicin)-cyclophosphamide, i.e. (TAC)-based) chemotherapy.

### TECHNICAL FIELD OF THE INVENTION

The present invention relates to methods and kits for the prediction of a likely outcome of chemotherapy in a cancer patient. More specifically, the invention relates to the prediction of tumour response to chemotherapy based on measurements of expression levels of a small set of marker genes. The set of marker genes is useful for the identification of breast cancer subtypes responsive to taxane based chemotherapy, such as e.g. a taxane-anthracycline-cyclophosphamine-based (e.g. Taxotere (docetaxel)-Adriamycin (doxorubicin)-cyclophosphamide, i.e. (TAC)-based) chemotherapy.

### BACKGROUND OF THE INVENTION

Breast cancer is one of the leading causes of cancer death in women in western countries. More specifically, breast cancer claims the lives of approximately 40,000 women and is diagnosed in approximately 200,000 women annually in the United States alone. Over the last few decades, adjuvant systemic therapy has led to markedly improved survival in early breast cancer (EBCTCG, 1998 a+b). This clinical experience has led to consensus recommendations offering adjuvant systemic therapy for the vast majority of breast cancer patients (Goldhirsch et al., 2003). In breast cancer, a multitude of treatment options are available which can be applied in addition to the routinely performed surgical removal of the tumour and subsequent radiation of the tumour bed.

Chemotherapy may be applied postoperative, i.e. in the adjuvant setting or preoperative, that is in the neoadjuvant setting in which patients receive several cycles of drug treatment over a limited period of time before remaining tumour cells are removed by surgery. In the past, neoadjuvant chemotherapy has been used for patients with locally advanced breast cancer. More recently, patients with large tumours are treated with neoadjuvant therapy as well. Primary goal is a reduction of tumour size in order to increase the possibility of breast-conserving treatment.

Yet, most if not all available drug treatments have numerous adverse effects which can severely impair patients' quality of life (Ganz et al., 2002). This makes it mandatory to select the treatment strategy on the basis of a careful risk assessment for the individual patient to avoid over-as well as under-treatment. Hence, it is desirable to have available a method for the prediction of the response of a patient to a particular chemotherapy prior to the actual onset of said chemotherapy. This allows for the best possible chemotherapeutic regimen to be selected for a particular patient. Folgueira et al. (2005, Clin. Cancer Res., 11(20), pp. 7434-7443) disclose a method for the prediction of the response of cancer patients to doxorubicin-based primary chemotherapy. Patients were classified in two groups, namely responders and non-responders. The classification is based on a trio of marker genes (PRSS11, MTSS1, CLPTM1) which correctly distinguished 95.4% of 44 samples analysed, with only two misclassifications. The classification is a single step classification. Folgueira et al., however, do not disclose marker genes or methods for the prediction of the response to taxane-based chemotherapy.

Ayers et al (2004, J. Clin. Oncology, 11(12), pp. 2284-2293) examine the feasibility of developing a multi-gene predictor of pathologic complete response to sequential weekly paclitaxel and fluorouracil+doxorubicin+cyclophosphamide (T/FAC) neoadjuvant chemotherapy for breast cancer. A multi-gene model with 74 marker genes was built. The authors conclude that transcriptional profiling has the potential to identify a gene expression pattern in breast cancer that may lead to clinically useful predictors of pathological complete response to T/FAC neoadjuvant therapy. The authors, however, do not disclose marker the specific combination of marker genes of the present invention.

WO 04/111603, assigned to Genomic Health Inc., discloses sets of genes the expression of which is useful for predicting whether cancer patients are likely to have beneficial treatment response to chemotherapy. Numerous marker genes are identified and used, alone or in combination with other marker genes, to predict breast cancer response. WO 04/111603, however, does not disclose a method for the prediction of the response of a breast cancer patient to taxane-based neoadjuvant chemotherapy using the specific combination of marker genes of the present invention.

Chang et al. (2003, The Lancet 362: 362-369) disclose a method for the prediction of therapeutic response to docetaxel (a taxane) in patients with breast cancer. Biopsy samples were taken in 24 patient before treatment and tumour response to neoadjuvant docetaxel treatment was assessed. 92 differentially expressed genes were identified, the expression of which correlated with tumour response. Based on the 92 differentially expressed genes, a predictor for tumour response was developed. Chang et al., however, do not disclose a predictor that uses the specific combination of marker genes of the present invention. Chang et al. also do not disclose a predictor that uses multiple binary classification steps.

Rouzier et al. (2005. PNAS, 102: 8315-8320) disclose a single marker gene of paclitaxel (a taxane) sensitivity in breast cancer. Rouzier et al., however, do not disclose a predictor that uses the specific combination of marker genes of the present invention, and they do not disclose a classification scheme with multiple binary classification steps.

Gianni et al. (2005, J. Clin. Oncol., 23: 7265-7277) disclose gene expression profiles in paraffin-embedded core biopsy tissue for the prediction of the response to a taxane-based chemotherapy. Gianni et al., however, do not disclose the specific combination of marker genes of the present invention; and they do not disclose a classification scheme with multiple binary classification steps.

Dressman et al. (2006, Clin. Cancer Res., 12: 819-826) disclose gene expression profiles that predict response to neoadjuvant taxane based chemotherapy. Dressmann et al, however, do not disclose the specific combination of marker genes of the present invention; and they do not disclose a classification scheme with multiple binary classification steps.

Thueringen et al. (2006, J. Clin. Oncol, 24: 1839-1845) disclose a gene signature for the prediction of pathological complete response to a taxane-based chemotherapy. Thueringen et al, however, do not disclose the specific combination of marker genes of the present invention; and they do not disclose a classification scheme with multiple binary classification steps.

US2005/0064455 of Baker et al. discloses gene expression markers for predicting response to taxane-based chemotherapy. This document, however, does not disclose the specific combination of marker genes of the present invention; and it does not disclose a classification scheme with multiple binary classification steps.

US2005/0266420 of Pusztai et al. discloses multi-gene predictors for the response to taxane-based chemotherapy. This document, however, does not disclose the specific combination of marker genes of the present invention; and it does not disclose a classification scheme with multiple binary classification steps.

US2006/0121511 of Lee et al. discloses biomarkers and methods for determining sensitivity to taxane-based chemotherapy. This document, however, does not disclose the specific combination of marker genes of the present invention; and it does not disclose a classification scheme with multiple binary classification steps. Furthermore, determining the sensitivity of a tumour to taxane-based chemotherapy requires the determination of the expression level of marker genes prior to and after administration of the chemotherapeutic to a sample of said tumour.

Tong et al. (2003, J. Chem. Inf. Comput. Sci. 43, 525-531) introduce combinations of decision trees referred to as “Decision Forests”. The method disclosed is used for the characterization and prediction of the binding affinity of chemical compounds to the estrogen receptor. The method uses multiple decision trees based upon simple “IF . . . THEN” rules based on a single descriptory value (attribute) or groups of descriptory values (attributes) used in a hierarchical manner where for each tree all attributes of all preceding trees are taken out to limit redundancy in prediction. A (linear) combination of the results then makes the final assessment to which class a given compound belongs. In later publications, this method is extended to proteomics with application to prostate cancer (Tong et al, Environmental Health Perspectives, 112 (16), 2004) and to genotyping applied to esophagial cancer (Xie et al., BMC Bioinformatics, 6 (2005)). The method and its variations disclosed in these publications deal with two-class problems only, whereas the algorithms provided in this invention allow for the prediction of an arbitrary number of classes, in the case at hand four classes are predicted.

Accurate prediction of the response of a breast cancer patient to taxane-based chemotherapy can help to select the most efficient and appropriate drug for breast cancer treatment in the patient, providing a means of individualized patient care. Thus, there is a need in the art for reliable methods of predicting the response of breast cancer patients to taxane-based neoadjuvant chemotherapy.

### SUMMARY OF THE INVENTION

The present invention is based on the unexpected finding that robust classification of breast tumour tissue samples into clinically relevant subgroups can be achieved by classifiers that use a small set of expression values of specific marker genes. The subgroups, as defined by the classification algorithm of the invention, represent taxane response classes which are characterized by a particular likelihood of tumour response to neoadjuvant taxane-based chemotherapy. Using the expression values of the small set of marker genes, a plurality of algorithms can be employed to perform the task of robust classification of an unknown sample into one of the response classes. Preferably, the taxane response class of a tumour is predicted hierarchically by separating a number of mutually disjoint aggregate or elementary classes at a time (cf. FIG. 1), i.e. by using a “classification tree” (decision tree). In each node of this tree, a partial classification is performed on the basis of a very small number of genes. Preferably, each separation step in the classification tree is achieved on the basis of the expression of a single specific marker gene, or a plurality of genes combined with a majority voting scheme. Each single marker gene can be substituted by further marker genes, provided the expression values of the further marker gene exhibit a high degree of correlation to the expression values of the marker gene. These genes are used to reliably distinguish aggregate and elementary classes in a classification tree until the sample can uniquely be assigned to its elementary class (the leaves of the tree structure).

Sets of marker genes are provided for the classification of a breast tumour into one of several breast cancer response classes. These sets of marker genes can be used to predict a patient's response to taxane-based chemotherapy, or to TAC-based chemotherapy, or to Taxotere-Adriamycin-Cyclophosphamide-based chemotherapy.

Hence the current invention provides means to decide—shortly after tumour biopsy—whether or not a certain mode of chemotherapy is likely to be beneficial to the patient's health and/or whether to maintain or change the applied mode of chemotherapy treatment.

Kits and devices for performing the above methods are further aspects of the invention.

### BRIEF DESCRIPTION OF THE FIGURES

FIG. 1: Decision tree for classification of breast cancer tissues into taxane response classes A, B, C, and D, based on marker gene expression measurements.

### DETAILED DESCRIPTION OF THE INVENTION

An “absolute expression level”, within the meaning of the invention, is understood as being the absolute expression level as obtained by using Affymetrix MASS algorithms and/or software package, which is well known to a person skilled in the art.

An “aggregate breast cancer response class”, within the meaning of the invention, shall be understood to be a breast cancer response class which comprises at least two sub-classes, each sub-class representing another aggregate or elementary breast cancer response class.

A “binary classification step”, within the meaning of the invention, is a classification step in which the members of a first group of patients/tumours is divided (classified) into two subgroups of patients/tumours of said first group of patients/tumours. The binary classification step can be based on measured expression levels of suitable marker genes.

A “breast cancer response class” within the meaning of the invention, shall be understood to be a group of breast cancer tumours showing a similar gene expression pattern and/or similar clinical behaviour. Preferably, the members of a “breast cancer response class” show, or are likely to show, a similar response to chemotherapy. The gene expression pattern and/or the clinical behaviour is preferably not similar to the gene expression pattern and/or the clinical behaviour of other tumours which do not belong to said breast cancer response class, i.e. the tumours belonging to one breast cancer response class are preferably distinguishable from tumours not belonging to said class.

The terms “cancer” and “cancerous” refer to or describe the physiological condition in mammals that is typically characterized by unregulated cell growth.

“Chemotherapy”, within this context, is understood to be the treatment of cancer with cytotoxic drugs.

“Classification” within the meaning of the invention, is understood to be the process of assigning a certain breast cancer response class to a given tumour. Classification can either be based on clinical information, or by applying a mathematical algorithm that utilizes clinical and/or gene expression data. Preferred classification methods of the invention are based on measurements of the expression of selected marker genes in a tumour sample.

A “correlation coefficient” between two variables, within the meaning of the invention, is understood to be the real number between −1 and 1 which measures the degree to which two variables are monotonely related. The correlation coefficient between two genes, within the context of the present application, shall be understood to be the correlation coefficient between the expression levels of said genes as determined in expression level measurements in multiple tissue samples. A high absolute correlation coefficient (i.e. negative signs disregarded) between two genes indicates that the two genes are co-regulated. In the following, correlation coefficient and correlation coefficient values shall be understood as being the absolute correlation coefficient values. A preferred correlation coefficient, within the context of the invention, is the “Pearson's Correlation Coefficient” known to any person skilled in the art.

“Determination of an expression level” of a gene in a tissue sample, within the meaning of the invention shall be understood to be any determination of the amount of mRNA coding for said gene, or a part of said gene, in said tissue sample; or any determination of the amount of the protein coded for by said gene in said tissue sample. Various methods to determine the expression level of a gene in a tissue are known in the art. These methods comprise, without limitation, PCR methods, real-time PCR methods, reverse transcriptase PCR methods, e.g. TaqMan RT-PCR, microarray experiments, immunohistochemistry (IHC), methods using the MassArray system of Sequenom, Inc. (San Diego, Calif.), SAGE Methods (Velculescu et al. 1995, Science 270, 484-487), the MPSS method of Brenner et al. (2000, Nature Biotechnology, A, pp. 630-634) and other methods known to the person skilled in the art.

An “elementary breast cancer response class”, within the meaning of the invention, shall be understood to be a group of breast cancer tumours having similar expression levels of certain marker genes and/or similar clinical behaviour. Elementary breast cancer response classes preferably comprise no further distinct breast cancer response classes within.

A “majority vote scheme”, within the meaning of the invention, uses a combination of two or more predictors to obtain a more robust classification. For a given unknown tissue sample, each predictor yields a “vote”, that is, a predicted response class. In a simple embodiment of this method with two possible response classes, the response class picked by the majority of the votes is predicted. In a more general and more advanced embodiment, votes are collected from all predictors and combined using a scalar (for example, real-valued), ordinal or logical mathematical function on these votes. This quantity is then compared to one or more threshold values to obtain a final predicted response class.

A “marker gene”, within the meaning of the invention, is any gene, the expression level of which is useful for the classification of a tumour sample into one of several aggregate or elementary breast cancer response classes, according to the invention.

A “microarray” within the meaning of the invention, shall be understood as being any type of solid support material, comprising a multitude of local features, each feature comprising immobilized nucleic acid probes. These nucleic acid probes are able to bind to free nucleic acids in a sample, wherein such binding can be detected by suitable methods. Various suitable technical implementations of microarrays are known to the person skilled in the art and commercially available. One well known example of a microarray is the GeneChip™ of Affymetrix, Inc. (Santa Clara, Calif.).

“Neoadjuvant therapy”, within the meaning of the invention, is adjunctive or adjuvant therapy given prior to the primary (main) therapy. Neoadjuvant therapy includes, for example, chemotherapy, radiation therapy, and hormone therapy. Neoadjuvant chemotherapy, e.g., is administered prior to surgery to shrink the tumour, so that surgery can be more effective, or, in the case of previously inoperable tumours, can be made possible.

“Prediction of the response to chemotherapy”, within the meaning of the invention, shall be understood to be the act of determining a likely outcome of a chemotherapy in a patient inflicted with cancer. The prediction of a response is preferably made with reference to probability values for reaching a desired or non-desired outcome of the chemotherapy. The predictive methods of the present invention can be used clinically to make treatment decisions by choosing the most appropriate treatment modalities for any particular patient.

A “previously known characteristic property” of a breast cancer response class is a property common to tumours or individuals of this class. This property may relate, e.g., to their response to chemotherapeutic treatment. Preferably, a previously known characteristic property may be expressed in terms of a probability that a tumour or individual of a breast cancer response class shows a certain response to chemotherapy.

The term “prognosis” is used herein to refer to the prediction of the likelihood of cancer-attributable death or progression, including recurrence and metastatic spread, of a neoplastic disease, such as breast cancer.

The “response of a tumour to chemotherapy”, within the meaning of the invention, relates to any response of the tumour to chemotherapy, preferably to a change in tumour mass and/or volume after initiation of neoadjuvant chemotherapy. Tumour response may be assessed in a neoadjuvant situation where the size of a tumour after systemic intervention can be compared to the initial size and dimensions as measured by CT, PET, mammogram, ultrasound or palpation. Response may also be assessed by caliper measurement or pathological examination of the tumour after biopsy or surgical resection. Response may be recorded in a quantitative fashion like percentage change in tumour volume or in a qualitative fashion like “clinical complete remission” (cCR), “clinical partial remission” (cPR), “clinical stable disease” (cSD), “clinical progressive disease” (cPD) or other qualitative criteria. Assessment of tumour response may be done early after the onset of neoadjuvant therapy e.g. after a few hours, days, weeks or preferably after a few months. A typical endpoint for response assessment is upon termination of neoadjuvant chemotherapy or upon surgical removal of residual tumour cells and/or the tumour bed. This is typically three month after initiation of neoadjuvant therapy.

A “Taxane”, within the meaning of the invention, can be Taxotere (docetaxel) or Taxol (paclitaxel).

A “tissue sample”, within the meaning of the invention, relates to tissue obtained from the human body by resection or biopsy which contains breast cancer cells. The tissue may originate from a carcinoma in situ, an invasive primary tumour, a recurrent tumour, lymph nodes infiltrated by tumour cells, or a metastatic lesion. The meaning of “tissue sample” is independent of the histological type of the primary tumour which may be an invasive ductal carcinoma, invasive lobular carcinoma, invasive tubular carcinoma, invasive medullar carcinoma, or invasive carcinoma of mixed type. After biopsy or resection, the breast tumour tissue may be preserved by storage in liquid nitrogen, dry ice or by fixation with appropriate reagents known in the field and subsequent embedding in paraffin wax. Preferably, tissue samples used in the present invention are already available, or are made available, prior to the start of the claimed methods. The detection of marker gene expression is not limited to the detection within a primary tumour, secondary tumour or metastatic lesion of breast cancer patients. It may also be detected in lymph nodes affected by breast cancer cells. In one embodiment of the invention, the sample to be analysed is tissue material from a neoplastic lesion taken by aspiration or punctuation, excision or by any other surgical method leading to biopsy or resected cellular material. The sample is preferably previously available. The step of taking the sample is preferably not part of the method. In one embodiment of the invention, the sample comprises cells obtained a breast cell “smear” collected, for example, by a nipple aspiration, ductal lavage, fine needle biopsy or from provoked or spontaneous nipple discharge. In another embodiment, the sample is a body fluid. Such fluids include, for example, blood fluids, lymph, ascitic fluids, gynecological fluids, or urine but not limited to these fluids.

The term “tumour,” as used herein, refers to all neoplastic cell growth and proliferation, whether malignant or benign, and all pre-cancerous and cancerous cells and tissues.

“Univariate classification”, within the meaning of the invention, is a classification of breast cancer tumours into two or more (aggregate or elementary) breast cancer response classes, based on the expression level of a single marker gene. Preferably, the classification comprises a comparison of the expression level of said marker gene with a predetermined threshold level.

Marker genes of the invention are defined either by their abbreviated gene name and by their ability to hybridise, i.e. to be detected, by probes defined in terms of their Affymetrix Probeset ID (see Tables 1 to 5b). Genes detected by a particular Affymetrix Probeset ID can be found at Affymetrix' homepage (http://www.affymetrix.com), or, more specific, at the HG U133A GeneChip Array Information Page on Affymetrix' homepage (http://www.affymetrix.com/support/technical/bvproduct.affx?product=hgu133) and other sources known to the person skilled in the art.

The current invention relates to a method for the prediction of the response of a breast cancer in a patient to a taxane-based chemotherapy, from a tumour sample of said patient, comprising steps of
(a) determining the expression level of a group of marker genes consisting of
   (i) a first marker gene selected from the group consisting of ESR1 and WARS and genes co-regulated thereto; and
   (ii) a second marker gene selected from the group consisting of CAV1, COL6A2 and UBE2C and genes co-regulated thereto; and
   (iii) a third marker gene selected from the group consisting of IGF1, FHL1, EFEMP1, IL6ST, SPARCL1, NET1, ISLR, ENO1 and CDH5 and genes co-regulated thereto;
(b) classifying said sample as belonging to one of several breast cancer response classes from said expression levels of said marker genes, wherein the outcome of said classification is dependent on the expression level of said first marker gene and on the expression level of at least one of said second or said third marker genes;
(c) predicting the response of said breast cancer in said patient to chemotherapy from previously known characteristic properties of tumours of said one of several breast cancer response classes.

Preferably, said first marker gene has a correlation coefficient with ESR1 or WARS of equal to or higher than 0.8 in Table 1; said second marker gene has a correlation coefficient with CAV1, COL6A2 or UBE2C of equal to or higher than 0.8 in Table 3; and said third marker gene has a correlation with IGF1, FHL1, EFEMP1, IL6ST, SPARCL1, NET1, ISLR, ENO1 or CDH5 of equal to or higher than 0.89 in Table 5a or Table 5b.

In another aspect of the invention, said first marker gene has a correlation coefficient with ESR1 or WARS of equal to or higher than 0.55 in Table 2; said second marker gene has a correlation coefficient with CAV1, COL6A2 or UBE2C of equal to or higher than 0.62 in Table 4; and said third marker gene has a correlation with IGF1, FHL1, EFEMP1, IL6ST, SPARCL1, NET1, ISLR, ENO1 or CDH5 of equal to or higher than 0.89 in Table 5a or Table 5b.

More preferably, said first marker gene is s ESR1 or WARS; and/or said second marker gene is CAV1, COL6A2 or UBE2C, and/or said third marker gene is IGF1, FHL1, EFEMP1, IL6ST, SPARCL1, NET1, ISLR, ENO1 or CDH5.

In preferred methods of the invention, said several breast cancer response classes are four breast cancer response classes. It is envisaged that four groups of breast cancer response classes are an optimal number of breast cancer response classes, because it allows for reliable classification and accurate prediction of the response of breast cancer tumours to taxane-based chemotherapy.

Methods of the invention predict the response of patients/tumours to taxane-based chemotherapy, more preferred to taxane-anthracycline-cyclophosphamide-based chemotherapy or to Taxotere-Adriamycin-cyclophosphamide-based chemotherapy.

In methods of the invention, said determining of the expression level is preferably in a sample taken before the onset of chemotherapy.

In preferred methods of the invention, said classification is based on a classification tree.

In preferred methods of the invention, said classification involves at least two binary classification steps.

In preferred methods of the invention, said classification step (b) is based on a mathematical discriminant function.

In preferred methods of the invention, said classification uses a k-nearest-neighbour (kNN) algorithm.

In preferred methods of the invention, said chemotherapy is a neoadjuvant chemotherapy.

In preferred methods of the invention, said response to chemotherapy is clinical response or pathological response.

In preferred methods of the invention, said patient is a human patient.

In preferred methods of the invention, said sample of a tumour is a fixed sample, a paraffin-embedded sample, a fresh sample, a fresh frozen sample or a frozen sample.

In preferred methods of the invention, said sample of a tumour is from fine needle biopsy, core biopsy or fine needle aspiration.

In preferred methods of the invention, said determination of the expression level is by microarray experiment, by RT-PCR, by SAGE, by immunohistochemistry, or by TaqMan.

The present invention further relates to a system for predicting the response to chemotherapy, of a breast cancer in a patient, comprising
(a) means for determining the expression level of a group of marker genes consisting of
   (i) a first marker gene selected from Table 1 or 2; and
   (ii) a second marker gene selected from Table 3 or 4; and
   (iii) a third marker gene selected from Table 5a or 5b;
(b) classification means, for automatically classifying said sample as belonging to one of several breast cancer response classes from said expression levels of said marker genes, wherein the outcome of said classification is dependent on the expression level of said first marker gene and on the expression level of at least one of said second or said third marker genes;
(c) prediction means for predicting the response of said breast cancer in said patient to chemotherapy from previously known characteristic properties of tumours of said one of several breast cancer response classes.

In preferred systems of the invention, said first marker gene is s ESR1 or WARS; said second marker gene is CAV1, COL6A2, or UBE2C, and said third marker gene is IGF1, FHL1, EFEMP1, IL6ST, SPARCL1, NET1, ISLR, ENO1, or CDH5.

In preferred systems of the invention, said several breast cancer response classes are four breast cancer response classes.

In preferred systems of the invention, said means for determining the expression level of a group of marker genes comprises a microarray, a system for 2D gel electrophoresis, a SAGE system or a system for immunohistochemical determination of expression levels.

In preferred systems of the invention, said system is adapted to perform a method of any of the above methods of the invention.

Methods of the invention use very small set of highly informative marker genes to classify a tumour sample as one out of several breast cancer response classes. It is envisaged that the above combinations of marker genes represent the smallest possible groups of marker genes that allow classification of tumour samples into relevant breast cancer response classes, that is, any algorithm depending on a genuine subset of genes will yield inferior results.

The person skilled in the art will readily appreciate that it is possible to substitute the expression level of any of the marker genes of the invention by the expression level of a co-regulated gene, said substitute expression level holding the same information as the expression level of the original marker gene.

Hence, the current invention further relates to a method of the above kind, wherein at least one marker gene of said group of marker genes is substituted by a substitute marker gene, said substitute marker gene being co-regulated with said at least one marker gene.

Preferably, said substitute marker gene has an absolute value of the correlation coefficient to the corresponding marker gene of equal to or higher than
(a) 0.5489 in Table 1 or Table 2, if said marker gene is ESR1 or WARS;
(b) 0.6241 in Table 3 or Table 4, if said marker gene is CAV1, UBE2C, or COL6A2; and
(c) 0.8946 in Table 5a or Table 5b, if said marker gene is IGF1, FHL1, EFEMP1, SLC, IL6ST, SPARCL1, NET1, ISLR, ENO1, or CDH5.

It is envisaged that these threshold values are appropriate for selecting substitute marker genes in methods of the invention. For calculation of these optimal threshold values, see Example 3.

Suitable substitute marker genes are identified by correlation coefficients listed in Tables 1-5b, because this provides a measure which is well defined and independent of the test cohort used to determine the correlation coefficients. These correlation coefficients are highly significant by construction and so may be verified in separate experiments. Alternatively, correlation coefficients determined from separate experiments can be used.

Alternative threshold values for the correlation coefficients in Tables 1-5b in methods of the invention are 0.6, preferably 0.7, 0.75, 0.8, 0.9, 0.95, 0.99, 0.999 or, most preferably 1.

According to the preferred invention, the classification scheme involves a decision tree with at least one majority voting step.

Other preferred methods of the invention use a k-nearest-neighbour (kNN) algorithm in the classification step. Alternatively, classification can be achieved using i. a. the following mathematical methods: Decision Trees, Random Forests, (weighted) k-Nearest Neighbours, Shrunken Centroids, Support Vector Machines, Majority Votes, Neural Networks, Self-Organizing Maps (SOM), Cohonen Maps, Principal Curves and Principal Surfaces, Generative Topographic Mapping (GTM). These methods are widely used and readily available to the person skilled in the art.

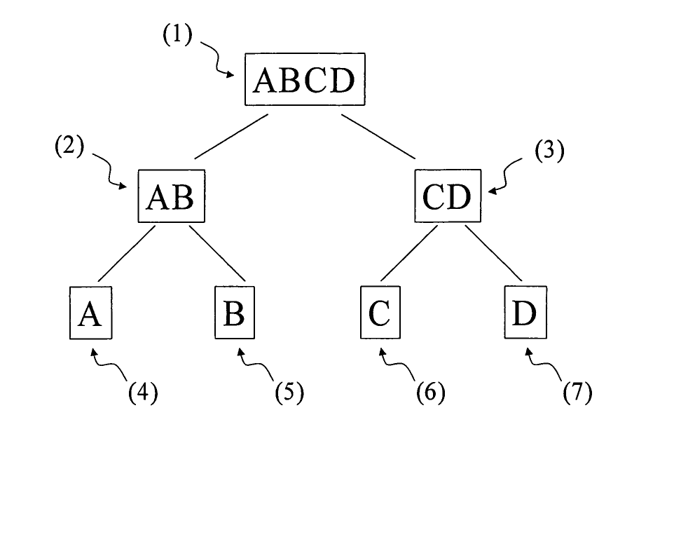

Preferred methods of the invention are methods comprising the steps of
(a) determining the expression level of at least one first marker gene in said sample of said tumour;
(b) classifying said sample as belonging to a first (FIG. 1, reference numeral 2) or a second (reference numeral 3) aggregate breast cancer response class from the expression level of said at least one first marker gene,
(c) determining the expression level of at least one second marker gene;
(d) classifying said sample as belonging to a first (4, 6) or a second (5, 7) elementary breast cancer response class of said first (2) or second (3) aggregate breast cancer response class from said expression level of said at least one second marker gene; and
(e) predicting the response of said breast cancer in said patient to chemotherapy from previously known characteristic properties of tumours of said first (4, 6) or second (5, 7) elementary breast cancer response class of said first (2) or second (3) aggregate breast cancer response class,
   wherein the choice of said at least one second marker gene is specific for (or alternatively, is dependent on) the aggregate breast cancer response class determined in step b).

The invention further relates to a method for the classification of a breast cancer tumour into clinically relevant breast cancer response classes, said method comprising steps of
(a) determining the expression level of at least one first marker gene in said sample of said tumour;
(b) classifying said sample as belonging to a first (2) or a second (3) aggregate breast cancer response class from the expression level of said at least one first marker gene,
(c) determining the expression level of at least one second marker gene; and
(d) classifying said sample as belonging to a first (4, 6) or a second (5, 7) elementary breast cancer response class of said first (2) or second (3) aggregate breast cancer response class from said expression level of said at least one second marker gene,
   wherein the choice of said at least one second marker gene is specific for the aggregate breast cancer response class determined in step b).

Preferably, the marker genes of the present inventions are used for classification.

According to a preferred embodiment of the invention, the step of determining the expression level of a marker gene is performed ex vivo.

Preferably, all method steps above are performed ex vivo. Furthermore, preferred methods comprise only method steps which are not performed on the human or animal body. Particularly preferred methods do not require the presence of the patient in any step of the method.

Determination of the expression levels of said at least one first and second marker gene is preferably done in parallel, e.g. on a microarray.

In a preferred method of the invention, said first classification step (b) is a univariate classification.

In preferred methods of the invention, if said tumour is classified to belong to the first elementary tumour class (4) of the first aggregate tumour class (2), the tumour is predicted to have a low likelihood of “pathological complete response” (i.e. 100% reduction in tumour mass), an intermediate likelihood of “partial response” (i.e. a reduction in tumour mass), and an intermediate likelihood of “no response” (i.e. no reduction in tumour mass), upon neoadjuvant taxane-based chemotherapy.

In preferred methods of the invention, if said tumour is classified to belong to the second elementary tumour class (5) of the first aggregate tumour class (2), the tumour is predicted to have an intermediate likelihood of “pathological complete response”, an intermediate likelihood of “partial response”, and a low likelihood of “no response”, upon neoadjuvant EC treatment.

In preferred methods of the invention, if said tumour is classified to belong to the first elementary tumour class (6) of the second aggregate tumour class (3), the tumour is predicted to have an intermediate likelihood of “pathological complete response”, a high likelihood of “partial response”, a and a low likelihood of “no response”, upon neoadjuvant EC treatment.

In preferred methods of the invention, if said tumour is classified to belong to the second elementary tumour class (7) of the second aggregate tumour class (3), the tumour is predicted to have a low likelihood of “pathological complete response”, a high likelihood of “partial response”, and a low likelihood of “no response”, upon neoadjuvant EC treatment.

A “low likelihood”, within the meaning of the invention, is preferably a likelihood p with 0≦p≦33%. An “intermediate likelihood”, within the meaning of the invention, is a likelihood p with 33%≦p≦66%. A “high likelihood”, within the meaning of the invention, is a likelihood p with 66%≦p≦100%.

Another aspect of the invention relates to methods for treating breast cancer in a patient, said method comprising one of the above methods of predicting the response of a breast cancer to chemotherapy, and applying said chemotherapy, if said breast cancer is predicted to show a sufficiently good response to said chemotherapy. A “sufficiently good response”, in this case, shall be a likelihood for pathological complete response of >20%, >50%, >80%, >90%, >95%, preferably >99%. According to another aspect of the invention, a “sufficiently good response” shall be understood as being a likelihood for partial response of >20%, >50%, >80%, >90%, >95%, preferably >99%.

The invention is further illustrated by way of the following examples. It shall be understood that the invention is not restricted to the specific embodiments described in the examples hereinafter.

### EXAMPLES

### Example 1

### Patient Selection, RNA Isolation from Tumour Tissue Biopsies and Gene Expression Measurement Utilizing HG-U133A Arrays of Affymetrix

Samples of primary breast carcinomas were available from 57 chemotherapy-naïve patients with operable (T2-3, N0-2) or locally advanced (T4a-d, N0-3) breast cancer were first treated with 2 cycles of TAC (docetaxel 75 mg/m2, doxorubicin 50 mg/m2, cyclophosphamide 500 mg/m2 Day 1, 3 weeks). All tumour samples were collected as needle biopsies of primary tumours prior to any treatment. The biopsies were obtained under local anaesthesia using Bardg MAGNUM™ Biopsy Instrument (C. R. Bard, Inc., Covington, US) with Bard® Magnum biopsy needles (BIP GmbH, Tuerkenfeld, Germany) following ultrasound guidance.

Total RNA was isolated from snap frozen breast tumour tissue biopsies. The tissue was crushed in liquid nitrogen, RLT-Buffer (QIAGEN, Hilden, Germany) was added and the homogenate spun through a QIAshredder column (QIAGEN, Hilden, Germany). From the eluate total RNA was isolated by the RNeasy Kit (QIAGEN, Hilden, Germany) according to the manufacturers instruction. RNA yield was determined by UV absorbance and RNA quality was assessed by analysis of ribosomal RNA band integrity on the Agilent Bioanalyzer (Palo Alto, Calif., USA).

Starting from 5 μg total RNA labelled cRNA was prepared for all 57 tumour samples using the one-cycle target labelling kit together with the appropriate control reagents (Affymetrix, Santa Clara, Calif., USA) according to the manufacturer's instruction. In brief, synthesis of first strand cDNA was done by a T7-linked oligo-dT primer, followed by second strand synthesis. Double-stranded cDNA product was purified and then used as template for an in vitro transcription reaction (IVT) in the presence of biotinylated UTP. Labelled cRNA was hybridised to HG-U133A arrays (Affymetrix, Santa Clara, Calif., USA) at 45° C. for 16 h in a hybridisation oven at a constant rotation (60 r.p.m.) and then washed and stained with a streptavidin-phycoerythrin conjugate using the GeneChip fluidic station. We scanned the arrays at 560 nm using the GeneArray Scanner G2500A from Hewlett Packard. The readings from the quantitative scanning were analysed using the Microarray Analysis Suit 5.0 (MAS 5.0) from Affymetrix. In the analysis settings the global scaling procedure was chosen which multiplied the output signal intensities of each array to a mean target intensity of 500. Routinely we obtained over 50 percent present calls per chip as calculated by MAS 5.0.

### Example 2

### Classification of Breast Tumour Tissues into Taxane Response Classes

For the separation of the aggregate breast cancer response classes AB and CD from ABCD (cf. FIG. 1) one of the following partial classifiers is used:
1. A majority voting scheme based on the expression level of ESR1 (Affymetrix Probeset ID 205225_at) and the expression values for the gene WARS (Affymetrix Probeset IDs 200628_s_at and 200629_at). Values of the ESR1 expression greater than 780 are considered a vote for aggregate breast cancer response class AB, values lower than the given threshold values are considered a vote for aggregate breast cancer response class CD. For the two WARS probeset IDs, the first is compared to 1060. Values less than this threshold are considered a vore a aggregate breast cancer response class AB, otherwise for CD. For the second probeset ID for WARS, values less than 1294 are considered a vote for aggregate breast cancer response class AB, otherwise for CD. The number of votes obtained from these three rules for aggregate breast cancer response class AB are the counted and compared against a threshold value of 0.5. If the number of votes for AB is higher than this value, the unknown tissue sample is predicted as a member of aggregate breast cancer response class AB, otherwise it is predicted as a member of aggregate breast cancer response class CD.
2. Alternatively, a predictor is based on a single of the three probeset IDs used in 1. The resulting aggregate breast cancer response class is then determined according to the result of the vote.

TABLE 1
Separation of Taxane Response Classes A, B vs. C, D
Genes with HIGH correlation coefficients (|cc| > 0.8)

| | ESR1 | WARS | WARS |
|---|---|---|---|
| | (205225_at) | (200628_s_at) | (200629_at) |
| CA12 (203963_at) | 0.88 | −0.64 | −0.63 |
| CA12 (204508_s_at) | 0.82 | −0.63 | −0.64 |
| CA12 (210735_s_at) | 0.84 | −0.55 | −0.61 |
| CA12 (214164_x_at) | 0.86 | −0.64 | −0.61 |
| CA12 (215867_x_at) | 0.88 | −0.64 | −0.62 |
| ERBB4 (214053_at) | 0.82 | −0.54 | −0.46 |
| ESR1 (205225_at) | 1.00 | −0.54 | −0.54 |
| GATA3 (209602_s_at) | 0.81 | −0.53 | −0.45 |
| GBP1 (202269_x_at) | −0.51 | 0.80 | 0.86 |
| GBP1 (202270_at) | −0.50 | 0.75 | 0.80 |
| KIAA0882 (212956_at) | 0.82 | −0.59 | −0.54 |
| KIAA0882 (212960_at) | 0.82 | −0.55 | −0.54 |
| PSMB9 (204279_at) | −0.39 | 0.82 | 0.78 |
| SLC39A6 (202088_at) | 0.82 | −0.54 | −0.55 |
| WARS (200628_s_at) | −0.54 | 1.00 | 0.86 |
| WARS (200629_at) | −0.54 | 0.86 | 1.00 |

TABLE 2
Separation of Taxane Response Classes A, B vs. C, D
Genes with SIGNIFICANT correlation coefficients (|cc| > 0.55)

| | ESR1 | WARS | WARS |
|---|---|---|---|
| | (205225_at) | (200628_s_at) | (200629_at) |
| — (206082_at) | −0.23 | 0.57 | 0.65 |
| — (211633_x_at) | −0.41 | 0.61 | 0.49 |
| — (211637_x_at) | −0.31 | 0.56 | 0.46 |
| — (211645_x_at) | −0.38 | 0.55 | 0.44 |
| — (213832_at) | 0.49 | −0.59 | −0.59 |
| — (216576_x_at) | −0.43 | 0.57 | 0.48 |
| — (217179_x_at) | −0.34 | 0.56 | 0.50 |
| — (217227_x_at) | −0.33 | 0.58 | 0.47 |
| — (217281_x_at) | −0.36 | 0.62 | 0.49 |
| — (221861_at) | 0.60 | −0.57 | −0.57 |
| — (43511_s_at) | 0.62 | −0.55 | −0.50 |
| ABAT (209459_s_at) | 0.60 | −0.60 | −0.52 |
| ABAT (209460_at) | 0.65 | −0.60 | −0.50 |
| ABCA3 (204343_at) | 0.57 | −0.51 | −0.54 |
| ACACB (43427_at) | 0.55 | −0.47 | −0.39 |
| ADAMDEC1 (206134_at) | −0.36 | 0.61 | 0.64 |
| ADCY9 (204497_at) | 0.67 | −0.59 | −0.60 |
| AK2 (205996_s_at) | −0.48 | 0.56 | 0.52 |
| AKR7A3 (206469_x_at) | 0.64 | −0.49 | −0.53 |
| AKR7A3 (216381_x_at) | 0.60 | −0.45 | −0.47 |
| AMD1 (201196_s_at) | −0.65 | 0.57 | 0.55 |
| ANP32E (221505_at) | −0.22 | 0.53 | 0.61 |
| ANXA9 (211712_s_at) | 0.76 | −0.54 | −0.45 |
| APBB2 (212985_at) | 0.68 | −0.58 | −0.58 |
| APOBEC3B (206632_s_at) | −0.57 | 0.44 | 0.46 |
| APOL6 (219716_at) | −0.28 | 0.58 | 0.62 |
| AR (211621_at) | 0.26 | −0.58 | −0.51 |
| ARF6 (203311_s_at) | −0.31 | 0.55 | 0.43 |
| ASS (207076_s_at) | −0.61 | 0.45 | 0.37 |
| ATP7B (204624_at) | 0.60 | −0.73 | −0.58 |
| AURKB (209464_at) | −0.53 | 0.52 | 0.58 |
| BAI2 (204966_at) | 0.52 | −0.58 | −0.54 |
| BCAS4 (220588_at) | 0.55 | −0.25 | −0.32 |
| BCL2 (203685_at) | 0.71 | −0.39 | −0.39 |
| BTN3A2 (209846_s_at) | −0.34 | 0.56 | 0.60 |
| BTN3A3 (204821_at) | −0.23 | 0.56 | 0.64 |
| BTN3A3 (38241_at) | −0.32 | 0.61 | 0.64 |
| BUB1B (203755_at) | −0.41 | 0.53 | 0.55 |
| C10orf116 (203571_s_at) | 0.59 | −0.51 | −0.55 |
| C10orf3 (218542_at) | −0.45 | 0.59 | 0.60 |
| C16orf34 (212109_at) | 0.48 | −0.57 | −0.54 |
| C18orf1 (207996_s_at) | 0.53 | −0.56 | −0.43 |
| C1orf34 (210652_s_at) | 0.65 | −0.58 | −0.53 |
| C1orf38 (207571_x_at) | −0.43 | 0.60 | 0.50 |
| C1QA (218232_at) | −0.30 | 0.63 | 0.48 |
| C1QB (202953_at) | −0.31 | 0.62 | 0.47 |
| C4A /// C4B (214428_x_at) | 0.53 | −0.55 | −0.43 |
| C6orf211 (218195_at) | 0.79 | −0.29 | −0.26 |
| C6orf75 (218877_s_at) | −0.36 | 0.59 | 0.63 |
| C8orf72 (221959_at) | 0.43 | −0.52 | −0.57 |
| CA12 (203963_at) | 0.88 | −0.64 | −0.63 |
| CA12 (204508_s_at) | 0.82 | −0.63 | −0.64 |
| CA12 (210735_s_at) | 0.84 | −0.55 | −0.61 |
| CA12 (214164_x_at) | 0.86 | −0.64 | −0.61 |
| CA12 (215867_x_at) | 0.88 | −0.64 | −0.62 |
| CACNA2D2 (204811_s_at) | 0.63 | −0.48 | −0.48 |
| CASP1 (209970_x_at) | −0.36 | 0.60 | 0.53 |
| CBFB (206788_s_at) | −0.69 | 0.70 | 0.53 |
| CCL18 (209924_at) | −0.54 | 0.55 | 0.42 |
| CCL5 (204655_at) | −0.25 | 0.60 | 0.57 |
| CCL8 (214038_at) | −0.36 | 0.57 | 0.49 |
| CCNA2 (203418_at) | −0.43 | 0.68 | 0.61 |
| CCNA2 (213226_at) | −0.38 | 0.58 | 0.59 |
| CCNB1 (214710_s_at) | −0.32 | 0.56 | 0.56 |
| CCNB2 (202705_at) | −0.49 | 0.59 | 0.61 |
| CCND1 (208711_s_at) | 0.57 | −0.21 | −0.32 |
| CCND1 (208712_at) | 0.64 | −0.38 | −0.48 |
| CCR5 (206991_s_at) | −0.19 | 0.59 | 0.48 |
| CD163 (203645_s_at) | −0.38 | 0.56 | 0.44 |
| CD3D (213539_at) | −0.23 | 0.58 | 0.52 |
| CD3Z (210031_at) | −0.24 | 0.64 | 0.59 |
| CD48 (204118_at) | −0.22 | 0.55 | 0.51 |
| CDC2 (203214_x_at) | −0.49 | 0.60 | 0.58 |
| CDC2 (210559_s_at) | −0.47 | 0.61 | 0.59 |
| CDC20 (202870_s_at) | −0.57 | 0.56 | 0.58 |
| CDC42EP1 (204693_at) | −0.57 | 0.19 | 0.16 |
| CDH3 (203256_at) | −0.57 | 0.43 | 0.36 |
| CDKN2A (207039_at) | −0.38 | 0.58 | 0.52 |
| CDKN2A (209644_x_at) | −0.47 | 0.59 | 0.56 |
| CELSR1 (41660_at) | 0.73 | −0.55 | −0.44 |
| CELSR2 (204029_at) | 0.56 | −0.33 | −0.30 |
| CELSR2 (36499_at) | 0.62 | −0.36 | −0.28 |
| CENPA (204962_s_at) | −0.54 | 0.63 | 0.62 |
| CHRD (211248_s_at) | 0.53 | −0.56 | −0.46 |
| CKS1B (201897_s_at) | −0.40 | 0.63 | 0.61 |
| CLIC4 (201559_s_at) | −0.59 | 0.59 | 0.40 |
| CLIC4 (221881_s_at) | −0.60 | 0.57 | 0.35 |
| CNAP1 (201774_s_at) | −0.45 | 0.62 | 0.64 |
| COPA (214336_s_at) | −0.47 | 0.59 | 0.44 |
| CORO1A (209083_at) | −0.27 | 0.61 | 0.47 |
| CORO1C (221676_s_at) | −0.69 | 0.66 | 0.57 |
| COTL1 (221059_s_at) | −0.56 | 0.61 | 0.57 |
| COX6C (201754_at) | 0.63 | −0.26 | −0.30 |
| COX7A1 (204570_at) | 0.30 | −0.44 | −0.58 |
| CRAT (209522_s_at) | 0.08 | −0.56 | −0.50 |
| CREB3L1 (213059_at) | 0.25 | −0.63 | −0.61 |
| CSDA (201161_s_at) | −0.56 | 0.43 | 0.44 |
| CSRP2 (211126_s_at) | −0.58 | 0.39 | 0.41 |
| CTSC (201487_at) | −0.55 | 0.71 | 0.66 |
| CTSS (202902_s_at) | −0.40 | 0.69 | 0.59 |
| CXCL10 (204533_at) | −0.28 | 0.62 | 0.59 |
| CXCR4 (209201_x_at) | −0.47 | 0.67 | 0.65 |
| CXCR4 (211919_s_at) | −0.48 | 0.63 | 0.60 |
| CXCR4 (217028_at) | −0.41 | 0.52 | 0.58 |
| CYBB (203922_s_at) | −0.38 | 0.69 | 0.57 |
| CYBB (203923_s_at) | −0.35 | 0.59 | 0.57 |
| CYP2B7P1 (206754_s_at) | 0.55 | −0.48 | −0.52 |
| DEK (200934_at) | −0.53 | 0.62 | 0.63 |
| DKC1 (216212_s_at) | −0.49 | 0.71 | 0.63 |
| DLG5 (201681_s_at) | 0.53 | −0.61 | −0.54 |
| DLG7 (203764_at) | −0.52 | 0.58 | 0.59 |
| DNAJC12 (218976_at) | 0.65 | −0.63 | −0.67 |
| DNALI1 (205186_at) | 0.70 | −0.51 | −0.48 |
| DNCH2 (219469_at) | 0.54 | −0.61 | −0.54 |
| DNMBP (212838_at) | 0.36 | −0.58 | −0.54 |
| DSC2 (204751_x_at) | −0.76 | 0.44 | 0.46 |
| DSCR1 (215253_s_at) | −0.58 | 0.31 | 0.30 |
| EDD1 (208883_at) | 0.55 | −0.27 | −0.25 |
| EGFL5 (212830_at) | 0.48 | −0.62 | −0.52 |
| EIF4G3 (201936_s_at) | 0.57 | −0.41 | −0.39 |
| ELOVL5 (208788_at) | 0.47 | −0.66 | −0.66 |
| EML2 (204398_s_at) | 0.71 | −0.35 | −0.32 |
| ENO1 (217294_s_at) | −0.62 | 0.63 | 0.43 |
| ENPP1 (205066_s_at) | 0.59 | −0.59 | −0.49 |
| EPS8L1 (91826_at) | 0.26 | −0.55 | −0.53 |
| ERBB4 (214053_at) | 0.82 | −0.54 | −0.46 |
| ESR1 (205225_at) | 1.00 | −0.54 | −0.54 |
| EVL (217838_s_at) | 0.78 | −0.44 | −0.40 |
| EZH1 (203249_at) | 0.57 | −0.35 | −0.35 |
| FAH (202862_at) | 0.36 | −0.61 | −0.58 |
| FAM64A (221591_s_at) | −0.60 | 0.51 | 0.55 |
| FBP1 (209696_at) | 0.65 | −0.40 | −0.38 |
| FCGR3A /// FCGR3B (204006_s_at) | −0.41 | 0.60 | 0.45 |
| FEN1 (204768_s_at) | −0.47 | 0.64 | 0.49 |
| FLJ20054 (219696_at) | 0.56 | −0.49 | −0.43 |
| FLJ20152 (218532_s_at) | 0.61 | −0.40 | −0.37 |
| FLJ20273 (218035_s_at) | 0.57 | −0.44 | −0.40 |
| FLJ20366 (218692_at) | 0.55 | −0.52 | −0.52 |
| FLNB (208614_s_at) | 0.65 | −0.32 | −0.40 |
| FN5 (219806_s_at) | −0.74 | 0.57 | 0.57 |
| FOXA1 (204667_at) | 0.60 | −0.67 | −0.63 |
| FOXC1 (213260_at) | −0.45 | 0.58 | 0.58 |
| FOXM1 (202580_x_at) | −0.48 | 0.61 | 0.58 |
| FPRL2 (214560_at) | −0.46 | 0.62 | 0.50 |
| FSCN1 (201564_s_at) | −0.55 | 0.52 | 0.46 |
| FXR1 (201635_s_at) | −0.34 | 0.60 | 0.47 |
| FXYD3 (202488_s_at) | 0.55 | −0.44 | −0.50 |
| GATA2 (209710_at) | 0.54 | −0.60 | −0.62 |
| GATA3 (209602_s_at) | 0.81 | −0.53 | −0.45 |
| GATA3 (209603_at) | 0.79 | −0.49 | −0.41 |
| GATA3 (209604_s_at) | 0.80 | −0.51 | −0.44 |
| GBP1 (202269_x_at) | −0.51 | 0.80 | 0.86 |
| GBP1 (202270_at) | −0.50 | 0.75 | 0.80 |
| GMNN (218350_s_at) | −0.49 | 0.57 | 0.58 |
| GMPS (214431_at) | −0.50 | 0.56 | 0.56 |
| GPD1L (212510_at) | 0.57 | −0.70 | −0.63 |
| GPSM2 (221922_at) | −0.33 | 0.53 | 0.57 |
| GRPR (207929_at) | 0.45 | −0.58 | −0.60 |
| GSTM3 (202554_s_at) | 0.50 | −0.50 | −0.60 |
| GTPBP4 (218238_at) | −0.34 | 0.61 | 0.59 |
| HCFC1R1 (218537_at) | 0.38 | −0.45 | −0.57 |
| HEBP1 (218450_at) | 0.55 | −0.35 | −0.36 |
| HHAT (219687_at) | 0.64 | −0.46 | −0.37 |
| HLA-C (211799_x_at) | −0.35 | 0.66 | 0.67 |
| HLA-DQA1 /// HLA-DQA2 | −0.36 | 0.59 | 0.45 |
| (212671_s_at) | | | |
| HLA-DRA (210982_s_at) | −0.33 | 0.58 | 0.43 |
| HLA-F (204806_x_at) | −0.42 | 0.67 | 0.70 |
| HLA-G (211530_x_at) | −0.35 | 0.68 | 0.67 |
| HMGB2 (208808_s_at) | −0.20 | 0.61 | 0.61 |
| IBRDC3 (36564_at) | −0.37 | 0.58 | 0.43 |
| ICA1 (207949_s_at) | 0.29 | −0.55 | −0.44 |
| ICA1 (210547_x_at) | 0.32 | −0.63 | −0.52 |
| ICAM1 (202637_s_at) | −0.44 | 0.58 | 0.48 |
| IFI16 (208965_s_at) | −0.34 | 0.62 | 0.57 |
| IFIH1 (219209_at) | −0.19 | 0.49 | 0.59 |
| IFIT2 (217502_at) | −0.34 | 0.56 | 0.48 |
| IFRG28 (219684_at) | −0.17 | 0.58 | 0.55 |
| IGF1R (203627_at) | 0.62 | −0.37 | −0.33 |
| IGF1R (203628_at) | 0.67 | −0.41 | −0.36 |
| IGFBP2 (202718_at) | 0.59 | −0.55 | −0.56 |
| IGFBP4 (201508_at) | 0.43 | −0.45 | −0.59 |
| IGH@ /// IGHA1 /// IGHA2 /// IGHD /// | −0.30 | 0.59 | 0.52 |
| IGHG1 /// IGHG2 /// IGHG3 /// IGHM | | | |
| /// MGC27165 /// LOC390714 | | | |
| (214916_x_at) | | | |
| IGHA1 /// IGHD /// IGHG1 /// IGHM /// | −0.34 | 0.56 | 0.47 |
| LOC390714 (216510_x_at) | | | |
| IGHA1 /// IGHG1 /// IGHG3 /// | −0.31 | 0.56 | 0.43 |
| LOC390714 (211868_x_at) | | | |
| IGHA1 /// IGHG1 /// IGHG3 /// | −0.34 | 0.62 | 0.49 |
| LOC390714 (216557_x_at) | | | |
| IGHD (214973_x_at) | −0.27 | 0.55 | 0.41 |
| IGHG1 /// MGC27165 (216542_x_at) | −0.31 | 0.57 | 0.50 |
| IGHM (211634_x_at) | −0.38 | 0.63 | 0.50 |
| IGHM (215949_x_at) | −0.35 | 0.63 | 0.54 |
| IGHM (216491_x_at) | −0.33 | 0.64 | 0.51 |
| IGHV1-69 (211635_x_at) | −0.32 | 0.64 | 0.55 |
| IGKC (211643_x_at) | −0.37 | 0.58 | 0.50 |
| IGKC (211644_x_at) | −0.33 | 0.58 | 0.45 |
| IGKC (216401_x_at) | −0.41 | 0.59 | 0.49 |
| IGKV1D-13 (216207_x_at) | −0.42 | 0.56 | 0.45 |
| IGLC2 (216984_x_at) | −0.30 | 0.55 | 0.42 |
| IGLJ3 (211798_x_at) | −0.34 | 0.59 | 0.47 |
| IGLJ3 (211881_x_at) | −0.34 | 0.59 | 0.47 |
| IGLV2-14 (217148_x_at) | −0.32 | 0.57 | 0.41 |
| IKBKB (209341_s_at) | 0.56 | −0.50 | −0.45 |
| IL15RA (207375_s_at) | −0.27 | 0.56 | 0.47 |
| IL2RG (204116_at) | −0.33 | 0.64 | 0.56 |
| IL6ST (204863_s_at) | 0.76 | −0.46 | −0.42 |
| IL6ST (211000_s_at) | 0.62 | −0.23 | −0.32 |
| IL6ST (212195_at) | 0.71 | −0.52 | −0.49 |
| IL6ST (212196_at) | 0.58 | −0.37 | −0.44 |
| ILF2 (200052_s_at) | −0.50 | 0.64 | 0.64 |
| IMPA2 (203126_at) | −0.60 | 0.53 | 0.46 |
| INDO (210029_at) | −0.49 | 0.76 | 0.76 |
| INPP4B (205376_at) | 0.49 | −0.55 | −0.47 |
| IRF1 (202531_at) | −0.35 | 0.70 | 0.71 |
| IRS1 (204686_at) | 0.73 | −0.55 | −0.57 |
| ISG20 (33304_at) | −0.33 | 0.69 | 0.65 |
| ITGB2 (202803_s_at) | −0.24 | 0.59 | 0.53 |
| ITM2C (221004_s_at) | −0.40 | 0.58 | 0.43 |
| ITPR1 (203710_at) | 0.61 | −0.32 | −0.32 |
| ITPR1 (211323_s_at) | 0.57 | −0.33 | −0.43 |
| JMJD2B (212492_s_at) | 0.72 | −0.67 | −0.61 |
| JMJD2B (212495_at) | 0.73 | −0.70 | −0.67 |
| JMJD2B (212496_s_at) | 0.72 | −0.64 | −0.59 |
| JMJD2B (215616_s_at) | 0.67 | −0.65 | −0.61 |
| KCTD3 (217894_at) | 0.57 | −0.58 | −0.49 |
| KIAA0020 (203712_at) | −0.44 | 0.62 | 0.63 |
| KIAA0040 (203144_s_at) | 0.60 | −0.45 | −0.40 |
| KIAA0746 (212311_at) | −0.53 | 0.66 | 0.59 |
| KIAA0746 (212314_at) | −0.52 | 0.67 | 0.60 |
| KIAA0882 (212956_at) | 0.82 | −0.59 | −0.54 |
| KIAA0882 (212960_at) | 0.82 | −0.55 | −0.54 |
| KIAA1324 (221874_at) | 0.55 | −0.65 | −0.57 |
| KIAA1467 (213234_at) | 0.66 | −0.42 | −0.38 |
| KIF20A (218755_at) | −0.44 | 0.53 | 0.55 |
| KIF2C (209408_at) | −0.48 | 0.55 | 0.56 |
| KIF2C (211519_s_at) | −0.57 | 0.57 | 0.56 |
| KIF4A (218355_at) | −0.51 | 0.55 | 0.60 |
| KIF5C (203130_s_at) | 0.72 | −0.44 | −0.46 |
| KRT6B (209126_x_at) | −0.57 | 0.50 | 0.43 |
| LAPTM5 (201720_s_at) | −0.44 | 0.62 | 0.47 |
| LASS6 (212442_s_at) | 0.68 | −0.49 | −0.45 |
| LBR (201795_at) | −0.46 | 0.62 | 0.67 |
| LDHB (201030_x_at) | −0.53 | 0.56 | 0.60 |
| LDHB (213564_x_at) | −0.51 | 0.52 | 0.58 |
| LGALS8 (208933_s_at) | 0.56 | −0.33 | −0.26 |
| LGALS8 (208935_s_at) | 0.61 | −0.41 | −0.37 |
| LILRB2 (207697_x_at) | −0.24 | 0.59 | 0.41 |
| LMNB1 (203276_at) | −0.42 | 0.57 | 0.59 |
| LMO4 (209205_s_at) | −0.66 | 0.46 | 0.52 |
| LOC339287 (212708_at) | 0.55 | −0.40 | −0.43 |
| LOC339562 (217480_x_at) | −0.40 | 0.62 | 0.49 |
| LOC391427 (217378_x_at) | −0.39 | 0.57 | 0.46 |
| LOC400451 (221880_s_at) | 0.51 | −0.64 | −0.56 |
| LOC492304 (202409_at) | 0.28 | −0.59 | −0.60 |
| LOC90355 (221823_at) | 0.67 | −0.49 | −0.40 |
| LOC91353 (215946_x_at) | −0.27 | 0.55 | 0.47 |
| LRRC17 (205381_at) | 0.48 | −0.61 | −0.53 |
| LRRC6 (206483_at) | 0.63 | −0.27 | −0.34 |
| LU (203009_at) | 0.67 | −0.73 | −0.69 |
| LU (40093_at) | 0.71 | −0.67 | −0.63 |
| LY6E (202145_at) | −0.49 | 0.56 | 0.53 |
| LYN (202625_at) | −0.41 | 0.57 | 0.48 |
| LYN (210754_s_at) | −0.45 | 0.60 | 0.45 |
| M6PR (200900_s_at) | −0.45 | 0.69 | 0.58 |
| MAD2L1 (203362_s_at) | −0.34 | 0.56 | 0.54 |
| MAGED2 (208682_s_at) | 0.63 | −0.63 | −0.59 |
| MAGED2 (213627_at) | 0.47 | −0.61 | −0.55 |
| MAGOH (210092_at) | −0.44 | 0.64 | 0.61 |
| MANSC1 (220945_x_at) | 0.19 | −0.55 | −0.41 |
| MAPT (203928_x_at) | 0.78 | −0.57 | −0.61 |
| MAPT (203929_s_at) | 0.79 | −0.55 | −0.58 |
| MAPT (206401_s_at) | 0.72 | −0.57 | −0.62 |
| MAST4 (210958_s_at) | 0.53 | −0.55 | −0.50 |
| MAST4 (40016_g_at) | 0.66 | −0.58 | −0.52 |
| MCAM (211042_x_at) | −0.58 | 0.50 | 0.63 |
| MCCC2 (209623_at) | 0.61 | −0.58 | −0.60 |
| MCF2L (212935_at) | 0.50 | −0.58 | −0.44 |
| MCL1 (200796_s_at) | −0.58 | 0.56 | 0.48 |
| MCM2 (202107_s_at) | −0.39 | 0.65 | 0.61 |
| MCM4 (222036_s_at) | −0.44 | 0.55 | 0.47 |
| MCM5 (201755_at) | −0.42 | 0.66 | 0.52 |
| MCM5 (216237_s_at) | −0.54 | 0.68 | 0.67 |
| MCM6 (201930_at) | −0.52 | 0.59 | 0.61 |
| MCM7 (208795_s_at) | −0.32 | 0.60 | 0.56 |
| MELK (204825_at) | −0.55 | 0.62 | 0.70 |
| METRN (219051_x_at) | 0.59 | −0.58 | −0.59 |
| MGC35048 (213392_at) | 0.55 | −0.59 | −0.57 |
| MGC39900 (214051_at) | −0.63 | 0.45 | 0.50 |
| MLPH (218211_s_at) | 0.66 | −0.67 | −0.61 |
| MRPS30 (222275_at) | 0.60 | −0.47 | −0.42 |
| MSH6 (211450_s_at) | −0.40 | 0.55 | 0.40 |
| MSX2 (210319_x_at) | 0.39 | −0.57 | −0.46 |
| MT1K (216336_x_at) | −0.30 | 0.59 | 0.53 |
| MT2A (212185_x_at) | −0.39 | 0.58 | 0.53 |
| MTUS1 (212096_s_at) | 0.38 | −0.56 | −0.53 |
| MYB (204798_at) | 0.66 | −0.39 | −0.29 |
| NASP (201970_s_at) | −0.48 | 0.62 | 0.63 |
| NAT1 (214440_at) | 0.67 | −0.59 | −0.56 |
| NBR1 (201384_s_at) | 0.62 | −0.36 | −0.42 |
| NEBL (203961_at) | 0.56 | −0.52 | −0.44 |
| NEIL1 (219396_s_at) | 0.35 | −0.57 | −0.46 |
| NME3 (204862_s_at) | 0.68 | −0.53 | −0.49 |
| NMI (203964_at) | −0.48 | 0.58 | 0.59 |
| NPDC1 (218086_at) | 0.44 | −0.63 | −0.62 |
| NPY1R (205440_s_at) | 0.69 | −0.30 | −0.31 |
| NUP155 (206550_s_at) | −0.38 | 0.59 | 0.52 |
| NUP93 (202188_at) | −0.42 | 0.55 | 0.43 |
| NUSAP1 (219978_s_at) | −0.39 | 0.63 | 0.58 |
| NUTF2 (202397_at) | −0.56 | 0.41 | 0.44 |
| OASL (205660_at) | −0.21 | 0.58 | 0.53 |
| PADI2 (209791_at) | −0.65 | 0.07 | 0.07 |
| PBX1 (212148_at) | 0.65 | −0.62 | −0.44 |
| PDGFD (219304_s_at) | 0.44 | −0.50 | −0.57 |
| PEX11A (205160_at) | 0.52 | −0.55 | −0.42 |
| PFKP (201037_at) | −0.62 | 0.57 | 0.61 |
| PGRMC2 (213227_at) | 0.56 | −0.48 | −0.46 |
| PHGDH (201397_at) | −0.68 | 0.46 | 0.54 |
| PIB5PA (213651_at) | 0.69 | −0.47 | −0.46 |
| PIGT (217770_at) | 0.57 | −0.40 | −0.37 |
| PLEK (203471_s_at) | −0.36 | 0.56 | 0.48 |
| PLK1 (202240_at) | −0.49 | 0.56 | 0.45 |
| PLOD1 (200827_at) | −0.59 | 0.33 | 0.29 |
| PLSCR1 (202446_s_at) | −0.32 | 0.64 | 0.61 |
| PPP1R3C (204284_at) | 0.54 | −0.56 | −0.51 |
| PRC1 (218009_s_at) | −0.41 | 0.57 | 0.62 |
| PRF1 (214617_at) | −0.48 | 0.67 | 0.63 |
| PRG1 (201858_s_at) | −0.38 | 0.62 | 0.47 |
| PROM1 (204304_s_at) | −0.57 | 0.37 | 0.46 |
| PSAT1 (220892_s_at) | −0.70 | 0.51 | 0.44 |
| PSF1 (206102_at) | −0.39 | 0.55 | 0.60 |
| PSMB10 (202659_at) | −0.27 | 0.60 | 0.56 |
| PSMB9 (204279_at) | −0.39 | 0.82 | 0.78 |
| PSME4 (212219_at) | −0.59 | 0.34 | 0.41 |
| PTPRC (207238_s_at) | −0.31 | 0.55 | 0.47 |
| PTPRC (212587_s_at) | −0.25 | 0.63 | 0.60 |
| PTPRM (203329_at) | 0.42 | −0.61 | −0.58 |
| PTTG1 (203554_x_at) | −0.40 | 0.65 | 0.61 |
| QDPR (209123_at) | 0.58 | −0.33 | −0.35 |
| RAB26 (219562_at) | 0.43 | −0.57 | −0.48 |
| RAD21 (200607_s_at) | −0.19 | 0.57 | 0.41 |
| RAP2A /// RAP2B (214487_s_at) | −0.58 | 0.52 | 0.35 |
| RARRES1 (206391_at) | −0.57 | 0.67 | 0.65 |
| RARRES1 (206392_s_at) | −0.55 | 0.69 | 0.64 |
| RARRES1 (221872_at) | −0.57 | 0.61 | 0.61 |
| RFC3 (204127_at) | −0.55 | 0.57 | 0.55 |
| RFC3 (204128_s_at) | −0.56 | 0.64 | 0.51 |
| RFC4 (204023_at) | −0.37 | 0.67 | 0.62 |
| RIPK2 (209545_s_at) | −0.58 | 0.54 | 0.51 |
| RPIA (212973_at) | −0.42 | 0.55 | 0.61 |
| RRM1 (201476_s_at) | −0.29 | 0.60 | 0.45 |
| RRM2 (209773_s_at) | −0.55 | 0.58 | 0.56 |
| RUNX1 (209360_s_at) | 0.45 | −0.70 | −0.62 |
| RUNX3 (204197_s_at) | −0.29 | 0.60 | 0.58 |
| RUNX3 (204198_s_at) | −0.40 | 0.69 | 0.66 |
| SAMHD1 (204502_at) | −0.27 | 0.67 | 0.58 |
| SCCPDH (201825_s_at) | 0.56 | −0.26 | −0.18 |
| SCCPDH (201826_s_at) | 0.70 | −0.50 | −0.41 |
| SCNN1A (203453_at) | 0.54 | −0.58 | −0.51 |
| SCUBE2 (219197_s_at) | 0.63 | −0.51 | −0.56 |
| SEMA3C (203789_s_at) | 0.56 | −0.48 | −0.49 |
| SEPHS1 (208941_s_at) | −0.43 | 0.55 | 0.52 |
| SERPINA3 (202376_at) | 0.55 | −0.40 | −0.32 |
| SERPINA5 (209443_at) | 0.56 | −0.46 | −0.37 |
| SH3BP4 (222258_s_at) | 0.32 | −0.56 | −0.52 |
| SIAH1 (221833_at) | 0.48 | −0.58 | −0.47 |
| SIAH1 (221834_at) | 0.49 | −0.58 | −0.52 |
| SIAH2 (209339_at) | 0.60 | −0.07 | −0.20 |
| SKP2 (203625_x_at) | −0.54 | 0.55 | 0.68 |
| SLC39A6 (202088_at) | 0.82 | −0.54 | −0.55 |
| SLC39A6 (202089_s_at) | 0.79 | −0.46 | −0.55 |
| SLC7A5 (201195_s_at) | −0.56 | 0.57 | 0.55 |
| SLC7A8 (202752_x_at) | 0.63 | −0.58 | −0.54 |
| SLC7A8 (216092_s_at) | 0.73 | −0.54 | −0.51 |
| SLC7A8 (216604_s_at) | 0.58 | −0.40 | −0.44 |
| SMC2L1 (204240_s_at) | −0.37 | 0.56 | 0.52 |
| SMC4L1 (201663_s_at) | −0.29 | 0.67 | 0.62 |
| SMC4L1 (201664_at) | −0.12 | 0.53 | 0.57 |
| SOD2 (215223_s_at) | −0.62 | 0.61 | 0.55 |
| SPDEF (213441_x_at) | 0.46 | −0.64 | −0.58 |
| SPDEF (214404_x_at) | 0.48 | −0.63 | −0.56 |
| SPDEF (220192_x_at) | 0.47 | −0.67 | −0.62 |
| SPFH2 (221543_s_at) | 0.61 | −0.37 | −0.32 |
| SPHK1 (219257_s_at) | −0.56 | 0.35 | 0.23 |
| SRD5A1 (204675_at) | −0.56 | 0.23 | 0.30 |
| SRD5A1 (210959_s_at) | −0.63 | 0.33 | 0.37 |
| STAT1 (209969_s_at) | −0.04 | 0.56 | 0.64 |
| STC2 (203438_at) | 0.70 | −0.41 | −0.41 |
| STIP1 (212009_s_at) | −0.43 | 0.57 | 0.45 |
| STMN1 (200783_s_at) | −0.61 | 0.53 | 0.54 |
| SYNCRIP (209024_s_at) | −0.62 | 0.58 | 0.64 |
| SYNCRIP (217834_s_at) | −0.56 | 0.50 | 0.55 |
| TAP1 (202307_s_at) | −0.36 | 0.71 | 0.78 |
| TAP2 (204769_s_at) | −0.31 | 0.53 | 0.56 |
| TAPBPL (218747_s_at) | −0.18 | 0.59 | 0.58 |
| TBPL1 (208398_s_at) | −0.49 | 0.39 | 0.60 |
| TCEAL1 (204045_at) | 0.65 | −0.58 | −0.50 |
| TFF1 (205009_at) | 0.72 | −0.44 | −0.42 |
| TFF3 (204623_at) | 0.66 | −0.47 | −0.41 |
| THBS4 (204776_at) | 0.51 | −0.49 | −0.55 |
| TJP3 (35148_at) | 0.48 | −0.66 | −0.56 |
| TMEM45A (219410_at) | −0.63 | 0.35 | 0.30 |
| TMPO (203432_at) | −0.26 | 0.59 | 0.53 |
| TMSL8 (205347_s_at) | −0.70 | 0.58 | 0.65 |
| TNFAIP8 (210260_s_at) | −0.54 | 0.64 | 0.52 |
| TNFRSF21 (214581_x_at) | −0.72 | 0.48 | 0.46 |
| TNFRSF21 (218856_at) | −0.65 | 0.31 | 0.29 |
| TPX2 (210052_s_at) | −0.43 | 0.60 | 0.58 |
| TRAM1 (201399_s_at) | −0.41 | 0.59 | 0.32 |
| TRBV19 /// TRBC1 (210915_x_at) | −0.25 | 0.57 | 0.51 |
| TRIM29 (202504_at) | −0.57 | 0.38 | 0.32 |
| TTMP (219474_at) | 0.61 | −0.51 | −0.45 |
| TUBA1 (212242_at) | −0.46 | 0.55 | 0.48 |
| TXNDC (208097_s_at) | −0.45 | 0.63 | 0.52 |
| UBE2I (208760_at) | 0.56 | −0.34 | −0.36 |
| UGCG (204881_s_at) | 0.60 | −0.33 | −0.40 |
| USP1 (202412_s_at) | −0.18 | 0.58 | 0.52 |
| VAV3 (218807_at) | 0.58 | −0.40 | −0.36 |
| WARS (200628_s_at) | −0.54 | 1.00 | 0.86 |
| WARS (200629_at) | −0.54 | 0.86 | 1.00 |
| WDR32 (219001_s_at) | 0.68 | −0.30 | −0.25 |
| WWTR1 (202134_s_at) | −0.62 | 0.42 | 0.36 |
| YBX1 (208627_s_at) | −0.75 | 0.67 | 0.60 |
| YES1 (202932_at) | −0.56 | 0.36 | 0.36 |
| ZNF42 (204139_x_at) | 0.67 | −0.50 | −0.48 |
| ZNF42 (210336_x_at) | 0.65 | −0.53 | −0.51 |
| ZNF446 (219900_s_at) | 0.56 | −0.55 | −0.50 |
| ZNF467 (214746_s_at) | 0.51 | −0.62 | −0.54 |
| ZNF91 (206059_at) | 0.57 | −0.55 | −0.49 |

For the separation of aggregate breast cancer tumor class AB into elementary breast cancer response classes A and B, one of the following partial classifiers is used:
1. A majority voting scheme bases on the expression values of three genes (four Affymetrix Probset IDs): First, the expression of genes CAV1 (with two Affymetrix Probeset IDs 203065_s_at and 212097_at), UBE2C (Affymetrix Probeset ID 202954_at), and COL6A2 (Affymetrix Probeset ID 213290_at) are measured. If the first probeset for CAV1, 203065_s_at, exceeds an expression value of 410, we count it as a vote for elementary breast cancer response class A, otherwise as a vote for B. If the second probeset for CAV1, 212097_at, exceeds 828, this counts as a vote for response class A, and for class B if this is not so. For UBE2C with its single probeset ID, we use a threshold value of 1724 to determine votes for A (if the UBE2C expression is below the given threshold value) or B (if its expression is above it). Finally, for high expression values of COL6A2 (i.e. above 643), a vote for A is counted, for values less than 643, a vote for B is counted. As above, the number of votes for A are counted and compared to a threshold value of 1.5. If there are less than 1.5 votes for A, the unknown sample is predicted as breast cancer response class B, and as breast cancer response class A otherwise.
2. Alternatively, a predictor is based on any single of the four probeset IDs used in 1. The resulting aggregate breast cancer response class is then determined according to the result of this vote

TABLE 3
Separation of Taxane Response Classes A vs. B
Genes with HIGH correlation coefficients (|cc| > 0.8)

| | CAV1 | CAV1 | UBE2C | COL6A2 |
|---|---|---|---|---|
| | (203065_s_at) | (212097_at) | (202954_at) | (213290_at) |
| AEBP1 (201792_at) | 0.40 | 0.44 | −0.19 | 0.84 |
| ANKRD25 (218418_s_at) | 0.72 | 0.83 | −0.47 | 0.71 |
| AQP1 (209047_at) | 0.76 | 0.81 | −0.56 | 0.28 |
| ASF1B (218115_at) | −0.39 | −0.49 | 0.84 | −0.34 |
| AURKB (209464_at) | −0.24 | −0.34 | 0.81 | −0.33 |
| BUB1B (203755_at) | −0.41 | −0.53 | 0.88 | −0.25 |
| C10orf3 (218542_at) | −0.49 | −0.57 | 0.82 | −0.27 |
| C10orf56 (212419_at) | 0.69 | 0.81 | −0.63 | 0.69 |
| C10orf56 (212423_at) | 0.69 | 0.83 | −0.68 | 0.71 |
| CAV1 (203065_s_at) | 1.00 | 0.92 | −0.52 | 0.66 |
| CAV1 (212097_at) | 0.92 | 1.00 | −0.61 | 0.66 |
| CAV2 (203323_at) | 0.74 | 0.82 | −0.55 | 0.66 |
| CAV2 (203324_s_at) | 0.82 | 0.87 | −0.54 | 0.66 |
| CCNA2 (203418_at) | −0.25 | −0.37 | 0.85 | −0.24 |
| CCNA2 (213226_at) | −0.29 | −0.35 | 0.80 | −0.22 |
| CCNB2 (202705_at) | −0.42 | −0.52 | 0.92 | −0.35 |
| CDKN3 (209714_s_at) | −0.44 | −0.54 | 0.88 | −0.45 |
| CENPA (204962_s_at) | −0.33 | −0.44 | 0.80 | −0.24 |
| COL1A1 (217430_x_at) | 0.52 | 0.49 | −0.38 | 0.82 |
| COL5A2 (221730_at) | 0.45 | 0.49 | −0.28 | 0.87 |
| COL6A1 (212091_s_at) | 0.52 | 0.50 | −0.26 | 0.87 |
| COL6A2 (213290_at) | 0.66 | 0.66 | −0.41 | 1.00 |
| CSPG2 (211571_s_at) | 0.53 | 0.54 | −0.35 | 0.81 |
| CSPG2 (215646_s_at) | 0.51 | 0.55 | −0.38 | 0.81 |
| CUGBP2 (202156_s_at) | 0.75 | 0.80 | −0.35 | 0.55 |
| DLG7 (203764_at) | −0.38 | −0.46 | 0.88 | −0.44 |
| ENPP2 (209392_at) | 0.82 | 0.86 | −0.45 | 0.42 |
| ESPL1 (38158_at) | −0.35 | −0.40 | 0.81 | −0.32 |
| FAP (209955_s_at) | 0.43 | 0.48 | −0.21 | 0.84 |
| FBN1 (202765_s_at) | 0.54 | 0.56 | −0.34 | 0.83 |
| FHL1 (201540_at) | 0.84 | 0.89 | −0.45 | 0.64 |
| FHL1 (210298_x_at) | 0.83 | 0.81 | −0.39 | 0.62 |
| FHL1 (214505_s_at) | 0.82 | 0.82 | −0.48 | 0.70 |
| FOXM1 (202580_x_at) | −0.37 | −0.48 | 0.81 | −0.27 |
| GLT8D2 (221447_s_at) | 0.66 | 0.66 | −0.44 | 0.81 |
| HMMR (207165_at) | −0.53 | −0.63 | 0.80 | −0.41 |
| HTRA1 (201185_at) | 0.61 | 0.69 | −0.44 | 0.83 |
| INHBA (210511_s_at) | 0.37 | 0.35 | −0.08 | 0.80 |
| KIAA0101 (202503_s_at) | −0.45 | −0.54 | 0.85 | −0.31 |
| KIF2C (209408_at) | −0.45 | −0.55 | 0.89 | −0.38 |
| LDB2 (206481_s_at) | 0.79 | 0.82 | −0.56 | 0.44 |
| LHFP (218656_s_at) | 0.80 | 0.86 | −0.67 | 0.63 |
| LOXL1 (203570_at) | 0.52 | 0.54 | −0.35 | 0.82 |
| MAD2L1 (203362_s_at) | −0.40 | −0.48 | 0.83 | −0.32 |
| MLF1IP (218883_s_at) | −0.40 | 0.48 | 0.84 | −0.36 |
| MYBL2 (201710_at) | −0.29 | −0.35 | 0.83 | −0.14 |
| NID1 (202008_s_at) | 0.70 | 0.73 | −0.48 | 0.87 |
| NUSAP1 (218039_at) | −0.39 | −0.45 | 0.86 | −0.32 |
| NUSAP1 (219978_s_at) | −0.52 | −0.59 | 0.84 | −0.35 |
| PCOLCE (202465_at) | 0.55 | 0.57 | −0.22 | 0.81 |
| PLSCR4 (218901_at) | 0.81 | 0.87 | −0.65 | 0.71 |
| PRC1 (218009_s_at) | −0.45 | −0.52 | 0.90 | −0.37 |
| PROS1 (207808_s_at) | 0.85 | 0.90 | −0.60 | 0.55 |
| PTTG1 (203554_x_at) | −0.44 | −0.51 | 0.85 | −0.39 |
| RACGAP1 (222077_s_at) | −0.37 | −0.45 | 0.83 | −0.28 |
| RRM2 (201890_at) | −0.28 | −0.40 | 0.82 | −0.19 |
| RRM2 (209773_s_at) | −0.41 | −0.53 | 0.87 | −0.26 |
| SPAG5 (203145_at) | −0.46 | −0.53 | 0.81 | −0.40 |
| SPARC (212667_at) | 0.62 | 0.69 | −0.50 | 0.86 |
| SPARCL1 (200795_at) | 0.74 | 0.82 | −0.64 | 0.66 |
| SPON1 (209436_at) | 0.58 | 0.65 | −0.50 | 0.82 |
| SRPX (204955_at) | 0.76 | 0.80 | −0.28 | 0.62 |
| STK6 (204092_s_at) | −0.35 | −0.42 | 0.80 | −0.28 |
| STK6 (208079_s_at) | −0.38 | −0.42 | 0.80 | −0.30 |
| THY1 (208850_s_at) | 0.44 | 0.48 | −0.34 | 0.82 |
| TNS1 (221748_s_at) | 0.82 | 0.88 | −0.48 | 0.60 |
| TOP2A (201292_at) | −0.37 | −0.44 | 0.81 | −0.33 |
| TPX2 (210052_s_at) | −0.34 | −0.44 | 0.88 | −0.26 |
| TRIP13 (204033_at) | −0.47 | −0.56 | 0.85 | −0.41 |
| TROAP (204649_at) | −0.33 | −0.43 | 0.87 | −0.32 |
| UBE2C (202954_at) | −0.52 | −0.61 | 1.00 | −0.41 |

TABLE 4
Separation of Taxane Response Groups A vs. B
Genes with SIGNIFICANT correlation coefficients (|cc| > 0.62)

| | CAV1 | CAV1 | UBE2C | COL6A2 |
|---|---|---|---|---|
| | (203065_s_at) | (212097_at) | (202954_at) | (213290_at) |
| — (212764_at) | 0.69 | 0.78 | −0.53 | 0.70 |
| — (213158_at) | 0.43 | 0.60 | −0.67 | 0.36 |
| — (222288_at) | 0.40 | 0.53 | −0.46 | 0.69 |
| ACACB (43427_at) | 0.51 | 0.63 | −0.46 | 0.22 |
| ACTG2 (202274_at) | 0.63 | 0.67 | −0.33 | 0.49 |
| ADAM12 (202952_s_at) | 0.41 | 0.47 | −0.25 | 0.76 |
| ADAM12 (213790_at) | 0.40 | 0.37 | −0.14 | 0.63 |
| ADAMTS2 (214454_at) | 0.32 | 0.35 | −0.18 | 0.71 |
| ADH1B (209612_s_at) | 0.73 | 0.72 | −0.39 | 0.39 |
| ADRA2A (209869_at) | 0.65 | 0.76 | −0.65 | 0.59 |
| AEBP1 (201792_at) | 0.40 | 0.44 | −0.19 | 0.84 |
| AKAP12 (210517_s_at) | 0.67 | 0.72 | −0.43 | 0.60 |
| AKT3 (212609_s_at) | 0.72 | 0.79 | −0.48 | 0.69 |
| ANGPTL2 (213001_at) | 0.62 | 0.69 | −0.24 | 0.76 |
| ANGPTL2 (213004_at) | 0.66 | 0.69 | −0.27 | 0.78 |
| ANGPTL2 (219514_at) | 0.56 | 0.59 | −0.31 | 0.70 |
| ANKRD25 (218418_s_at) | 0.72 | 0.83 | −0.47 | 0.71 |
| ANXA1 (201012_at) | 0.77 | 0.76 | −0.32 | 0.66 |
| APOBEC3B (206632_s_at) | −0.40 | −0.49 | 0.66 | −0.24 |
| AQP1 (207542_s_at) | 0.64 | 0.67 | −0.43 | 0.29 |
| AQP1 (209047_at) | 0.76 | 0.81 | −0.56 | 0.28 |
| ASF1B (218115_at) | −0.39 | −0.49 | 0.84 | −0.34 |
| ASPM (219918_s_at) | −0.31 | −0.43 | 0.74 | −0.23 |
| ASPN (219087_at) | 0.30 | 0.38 | −0.41 | 0.66 |
| AURKB (209464_at) | −0.24 | −0.34 | 0.81 | −0.33 |
| BGN (201261_x_at) | 0.53 | 0.63 | −0.31 | 0.76 |
| BIRC5 (202094_at) | −0.09 | −0.23 | 0.69 | −0.30 |
| BIRC5 (202095_s_at) | −0.16 | −0.30 | 0.72 | −0.28 |
| BUB1B (203755_at) | −0.41 | −0.53 | 0.88 | −0.25 |
| C10orf10 (209183_s_at) | 0.74 | 0.76 | −0.35 | 0.49 |
| C10orf3 (218542_at) | −0.49 | −0.57 | 0.82 | −0.27 |
| C10orf56 (212419_at) | 0.69 | 0.81 | −0.63 | 0.69 |
| C10orf56 (212423_at) | 0.69 | 0.83 | −0.68 | 0.71 |
| C1QR1 (202878_s_at) | 0.74 | 0.77 | −0.47 | 0.38 |
| C1S (208747_s_at) | 0.71 | 0.73 | −0.40 | 0.77 |
| C3 (217767_at) | 0.70 | 0.66 | −0.61 | 0.44 |
| CAT (211922_s_at) | 0.63 | 0.62 | −0.35 | 0.40 |
| CAV1 (203065_s_at) | 1.00 | 0.92 | −0.52 | 0.66 |
| CAV1 (212097_at) | 0.92 | 1.00 | −0.61 | 0.66 |
| CAV2 (203323_at) | 0.74 | 0.82 | −0.55 | 0.66 |
| CAV2 (203324_s_at) | 0.82 | −0.87 | −0.54 | 0.66 |
| CCL14 /// CCL15 | 0.60 | 0.70 | −0.49 | 0.20 |
| (205392_s_at) | | | | |
| CCNA2 (203418_at) | −0.25 | −0.37 | 0.85 | −0.24 |
| CCNA2 (213226_at) | −0.29 | −0.35 | 0.80 | −0.22 |
| CCNB1 (214710_s_at) | −0.39 | −0.47 | 0.79 | −0.30 |
| CCNB2 (202705_at) | −0.42 | −0.52 | 0.92 | −0.35 |
| CD248 (219025_at) | 0.41 | 0.54 | −0.28 | 0.67 |
| CDC2 (203213_at) | −0.45 | −0.53 | 0.77 | −0.44 |
| CDC2 (203214_x_at) | −0.34 | −0.44 | 0.76 | −0.34 |
| CDC2 (210559_s_at) | −0.38 | −0.49 | 0.80 | −0.32 |
| CDC20 (202870_s_at) | −0.44 | −0.54 | 0.80 | −0.36 |
| CDH11 (207172_s_at) | 0.37 | 0.39 | −0.23 | 0.72 |
| CDH11 (207173_x_at) | 0.43 | 0.48 | −0.29 | 0.73 |
| CDH5 (204677_at) | 0.70 | 0.75 | −0.46 | 0.39 |
| CDKN1C (213348_at) | 0.72 | 0.71 | −0.62 | 0.44 |
| CDKN3 (209714_s_at) | −0.44 | −0.54 | 0.88 | −0.45 |
| CENPA (204962_s_at) | −0.33 | −0.44 | 0.80 | −0.24 |
| CENPF (207828_s_at) | −0.31 | −0.37 | 0.66 | −0.22 |
| CFH (213800_at) | 0.76 | 0.79 | −0.33 | 0.45 |
| CHST3 (32094_at) | 0.42 | 0.52 | −0.21 | 0.64 |
| CIDEC (219398_at) | 0.59 | 0.63 | −0.23 | 0.23 |
| CKS1B (201897_s_at) | −0.48 | −0.57 | 0.66 | −0.29 |
| CKS2 (204170_s_at) | −0.67 | −0.73 | 0.76 | −0.41 |
| CLEC11A (205131_x_at) | 0.42 | 0.51 | −0.44 | 0.63 |
| CNAP1 (201774_s_at) | −0.24 | −0.32 | 0.71 | −0.25 |
| CNN1 (203951_at) | 0.54 | 0.63 | −0.49 | 0.55 |
| COL10A1 (205941_s_at) | 0.27 | 0.26 | −0.04 | 0.65 |
| COL10A1 (217428_s_at) | 0.28 | 0.28 | −0.09 | 0.68 |
| COL14A1 (212865_s_at) | 0.63 | 0.72 | −0.75 | 0.61 |
| COL15A1 (203477_at) | 0.66 | 0.61 | −0.29 | 0.55 |
| COL16A1 (204345_at) | 0.47 | 0.61 | −0.66 | 0.66 |
| COL18A1 (209081_s_at) | 0.43 | 0.45 | −0.25 | 0.71 |
| COL1A1 (202311_s_at) | 0.06 | 0.09 | −0.17 | 0.66 |
| COL1A1 (217430_x_at) | 0.52 | 0.49 | −0.38 | 0.82 |
| COL5A1 (203325_s_at) | 0.25 | 0.34 | −0.18 | 0.74 |
| COL5A1 (212488_at) | 0.39 | 0.49 | −0.25 | 0.79 |
| COL5A1 (212489_at) | 0.34 | 0.39 | −0.21 | 0.78 |
| COL5A2 (221730_at) | 0.45 | 0.49 | −0.28 | 0.87 |
| COL5A3 (218975_at) | 0.24 | 0.25 | −0.12 | 0.65 |
| COL6A1 (212091_s_at) | 0.52 | 0.50 | −0.26 | 0.87 |
| COL6A1 (212937_s_at) | 0.47 | 0.45 | −0.29 | 0.69 |
| COL6A1 (212940_at) | 0.44 | 0.41 | −0.36 | 0.63 |
| COL6A2 (213290_at) | 0.66 | 0.66 | −0.41 | 1.00 |
| COL8A2 (221900_at) | 0.38 | 0.41 | −0.26 | 0.77 |
| COL8A2 (52651_at) | 0.34 | 0.34 | −0.29 | 0.74 |
| COPG (217749_at) | −0.64 | −0.59 | 0.44 | −0.29 |
| COPZ2 (219561_at) | 0.43 | 0.51 | −0.37 | 0.72 |
| COX7A1 (204570_at) | 0.59 | 0.73 | −0.40 | 0.65 |
| CPA3 (205624_at) | 0.57 | 0.53 | −0.47 | 0.71 |
| CPT1A (203633_at) | −0.27 | −0.27 | 0.67 | −0.35 |
| CRIM1 (202551_s_at) | 0.20 | 0.31 | −0.62 | 0.00 |
| CRISPLD2 (221541_at) | 0.60 | 0.67 | −0.42 | 0.77 |
| CRYAB (209283_at) | 0.73 | 0.77 | −0.50 | 0.55 |
| CSPG2 (204619_s_at) | 0.40 | 0.51 | −0.38 | 0.74 |
| CSPG2 (211571_s_at) | 0.53 | 0.54 | −0.35 | 0.81 |
| CSPG2 (215646_s_at) | 0.51 | 0.55 | −0.38 | 0.81 |
| CSPG2 (221731_x_at) | 0.43 | 0.57 | −0.38 | 0.68 |
| CTGF (209101_at) | 0.60 | 0.58 | −0.27 | 0.68 |
| CTSL2 (210074_at) | −0.17 | −0.23 | 0.63 | 0.05 |
| CTTN (201059_at) | −0.38 | −0.34 | 0.65 | −0.21 |
| CUGBP2 (202156_s_at) | 0.75 | 0.80 | −0.35 | 0.55 |
| CXCL12 (203666_at) | 0.72 | 0.71 | −0.37 | 0.57 |
| CXCL12 (209687_at) | 0.75 | 0.73 | −0.43 | 0.57 |
| CYP1B1 (202437_s_at) | 0.55 | 0.56 | −0.42 | 0.63 |
| DAB2 (201279_s_at) | 0.62 | 0.61 | −0.30 | 0.72 |
| DAB2 (201280_s_at) | 0.73 | 0.78 | −0.48 | 0.77 |
| DCN (209335_at) | 0.68 | 0.75 | −0.50 | 0.77 |
| DCN (211813_x_at) | 0.71 | 0.70 | −0.43 | 0.74 |
| DCN (211896_s_at) | 0.75 | 0.74 | −0.48 | 0.78 |
| DDR2 (205168_at) | 0.47 | 0.57 | −0.36 | 0.63 |
| DF (205382_s_at) | 0.71 | 0.72 | −0.49 | 0.52 |
| DIXDC1 (214724_at) | 0.62 | 0.64 | −0.71 | 0.37 |
| DKK3 (202196_s_at) | 0.54 | 0.56 | −0.32 | 0.67 |
| DKK3 (214247_s_at) | 0.49 | 0.54 | −0.26 | 0.70 |
| DLC1 (210762_s_at) | 0.77 | 0.75 | −0.54 | 0.63 |
| DLG7 (203764_at) | −0.38 | −0.46 | 0.88 | −0.44 |
| DPT (207977_s_at) | 0.69 | 0.68 | −0.29 | 0.58 |
| DPT (213068_at) | 0.69 | 0.71 | −0.29 | 0.59 |
| DPT (213071_at) | 0.66 | 0.72 | −0.34 | 0.50 |
| DPYSL3 (201430_s_at) | 0.32 | 0.36 | −0.25 | 0.68 |
| DPYSL3 (201431_s_at) | 0.50 | 0.59 | −0.32 | 0.77 |
| DTL (218585_s_at) | −0.34 | −0.39 | 0.65 | −0.41 |
| ECT2 (219787_s_at) | −0.47 | −0.53 | 0.76 | −0.39 |
| EDG2 (204036_at) | 0.58 | 0.63 | −0.43 | 0.63 |
| EFEMP1 (201842_s_at) | 0.66 | 0.68 | −0.50 | 0.56 |
| EHD2 (221870_at) | 0.75 | 0.77 | −0.61 | 0.70 |
| EIF4EBP1 (221539_at) | −0.54 | −0.64 | 0.42 | −0.36 |
| ELN (212670_at) | 0.51 | 0.58 | −0.62 | 0.64 |
| EMILIN1 (204163_at) | 0.54 | 0.60 | −0.26 | 0.79 |
| ENPP2 (209392_at) | 0.82 | 0.86 | −0.45 | 0.42 |
| ENPP2 (210839_s_at) | 0.77 | 0.80 | −0.31 | 0.39 |
| EPAS1 (200878_at) | 0.73 | 0.72 | −0.53 | 0.31 |
| ESPL1 (204817_at) | −0.34 | −0.44 | 0.77 | −0.16 |
| ESPL1 (38158_at) | −0.35 | −0.40 | 0.81 | −0.32 |
| F13A1 (203305_at) | 0.58 | 0.56 | −0.22 | 0.63 |
| F2R (203989_x_at) | 0.55 | 0.59 | −0.33 | 0.63 |
| FABP4 (203980_at) | 0.67 | 0.66 | −0.28 | 0.44 |
| FABP5 (202345_s_at) | 0.60 | 0.67 | −0.33 | 0.69 |
| FAM64A (221591_s_at) | −0.10 | −0.16 | 0.71 | −0.18 |
| FAP (209955_s_at) | 0.43 | 0.48 | −0.21 | 0.84 |
| FAS (204780_s_at) | 0.61 | 0.63 | −0.44 | 0.66 |
| FAS (215719_x_at) | 0.60 | 0.59 | −0.53 | 0.68 |
| FAS (216252_x_at) | 0.66 | 0.65 | −0.39 | 0.70 |
| FBLN1 (202994_s_at) | 0.71 | 0.77 | −0.70 | 0.75 |
| FBLN1 (202995_s_at) | 0.60 | 0.61 | −0.54 | 0.77 |
| FBLN2 (203886_s_at) | 0.56 | 0.60 | −0.44 | 0.67 |
| FBN1 (202765_s_at) | 0.54 | 0.56 | −0.34 | 0.83 |
| FBN1 (202766_s_at) | 0.51 | 0.63 | −0.39 | 0.78 |
| FEN1 (204768_s_at) | −0.22 | −0.41 | 0.71 | −0.16 |
| FHL1 (201539_s_at) | 0.70 | 0.72 | −0.30 | 0.57 |
| FHL1 (201540_at) | 0.84 | 0.89 | −0.45 | 0.64 |
| FHL1 (210298_x_at) | 0.83 | 0.81 | −0.39 | 0.62 |
| FHL1 (214505_s_at) | 0.82 | 0.82 | −0.48 | 0.70 |
| FLJ10357 (220326_s_at) | 0.60 | 0.68 | −0.39 | 0.69 |
| FLJ10357 (58780_s_at) | 0.65 | 0.78 | −0.41 | 0.71 |
| FLRT2 (204359_at) | 0.70 | 0.78 | −0.56 | 0.57 |
| FMO2 (211726_s_at) | 0.79 | 0.76 | −0.35 | 0.49 |
| FMOD (202709_at) | 0.70 | 0.73 | −0.49 | 0.55 |
| FOXM1 (202580_x_at) | −0.37 | −0.48 | 0.81 | −0.27 |
| G0S2 (213524_s_at) | 0.64 | 0.65 | −0.19 | 0.38 |
| GAS1 (204457_s_at) | 0.58 | 0.65 | −0.32 | 0.77 |
| GAS6 (202177_at) | 0.76 | 0.74 | −0.39 | 0.68 |
| GAS7 (202191_s_at) | 0.63 | 0.71 | −0.43 | 0.67 |
| GAS7 (211067_s_at) | 0.50 | 0.53 | −0.39 | 0.72 |
| GBE1 (203282_at) | 0.59 | 0.66 | −0.31 | 0.49 |
| GEM (204472_at) | 0.63 | 0.66 | −0.42 | 0.58 |
| GIMAP6 (219777_at) | 0.73 | 0.70 | −0.53 | 0.28 |
| GLT8D2 (221447_s_at) | 0.66 | 0.66 | −0.44 | 0.81 |
| GMNN (218350_s_at) | −0.24 | −0.33 | 0.70 | −0.32 |
| GPR116 (212950_at) | 0.68 | 0.75 | −0.42 | 0.29 |
| GPX3 (201348_at) | 0.65 | 0.64 | −0.20 | 0.37 |
| GPX3 (214091_s_at) | 0.63 | 0.60 | −0.16 | 0.31 |
| GRP (206326_at) | 0.35 | 0.42 | −0.46 | 0.68 |
| GTSE1 (204318_s_at) | −0.32 | −0.42 | 0.75 | −0.28 |
| HIST1H4C (205967_at) | −0.17 | −0.26 | 0.64 | −0.35 |
| HMGB3 (203744_at) | −0.51 | −0.56 | 0.63 | −0.30 |
| HMMR (207165_at) | −0.53 | −0.63 | 0.80 | −0.41 |
| HN1 (217755_at) | −0.60 | −0.67 | 0.77 | −0.25 |
| HOXA5 (213844_at) | 0.65 | 0.64 | −0.33 | 0.32 |
| HTRA1 (201185_at) | 0.61 | 0.69 | −0.44 | 0.83 |
| ICAM2 (204683_at) | 0.65 | 0.64 | −0.21 | 0.38 |
| ICT1 (204868_at) | −0.51 | −0.59 | 0.63 | −0.40 |
| ID4 (209291_at) | 0.68 | 0.72 | −0.48 | 0.46 |
| IGF1 (209540_at) | 0.70 | 0.70 | −0.60 | 0.44 |
| IGF1 (209541_at) | 0.69 | 0.74 | −0.65 | 0.52 |
| IGF1 (209542_x_at) | 0.70 | 0.75 | −0.63 | 0.39 |
| IL1R1 (202948_at) | 0.54 | 0.64 | −0.54 | 0.53 |
| INHBA (210511_s_at) | 0.37 | 0.35 | −0.08 | 0.80 |
| ISLR (207191_s_at) | 0.29 | 0.34 | −0.14 | 0.77 |
| ITGBL1 (214927_at) | 0.67 | 0.71 | −0.45 | 0.59 |
| ITIH5 (219064_at) | 0.71 | 0.76 | −0.48 | 0.48 |
| ITM2A (202746_at) | 0.60 | 0.64 | −0.60 | 0.33 |
| ITM2A (202747_s_at) | 0.70 | 0.72 | −0.63 | 0.36 |
| JAM2 (219213_at) | 0.75 | 0.80 | −0.43 | 0.43 |
| KCTD12 (212188_at) | 0.75 | 0.75 | −0.53 | 0.48 |
| KIAA0101 (202503_s_at) | −0.45 | −0.54 | 0.85 | −0.31 |
| KIF20A (218755_at) | −0.37 | −0.45 | 0.78 | −0.17 |
| KIF2C (209408_at) | −0.45 | −0.55 | 0.89 | −0.38 |
| KIF2C (211519_s_at) | −0.29 | −0.37 | 0.76 | −0.19 |
| KIF4A (218355_at) | −0.44 | −0.56 | 0.77 | −0.51 |
| KIFC1 (209680_s_at) | −0.31 | −0.41 | 0.77 | −0.46 |
| KIT (205051_s_at) | 0.61 | 0.64 | −0.43 | 0.52 |
| KPNA2 (201088_at) | −0.36 | −0.42 | 0.71 | −0.26 |
| KPNA2 (211762_s_at) | −0.37 | −0.45 | 0.63 | −0.32 |
| LAMA2 (205116_at) | 0.56 | 0.65 | −0.63 | 0.62 |
| LDB2 (206481_s_at) | 0.79 | 0.82 | −0.56 | 0.44 |
| LHFP (218656_s_at) | 0.80 | 0.86 | −0.67 | 0.63 |
| LMNB1 (203276_at) | −0.45 | −0.53 | 0.67 | −0.23 |
| LMOD1 (203766_s_at) | 0.71 | 0.76 | −0.59 | 0.53 |
| LOC146909 (222039_at) | −0.29 | −0.40 | 0.75 | −0.33 |
| LOC492304 (202409_at) | 0.52 | 0.66 | −0.53 | 0.63 |
| LOX (204298_s_at) | 0.50 | 0.48 | −0.21 | 0.69 |
| LOX (215446_s_at) | 0.44 | 0.53 | −0.26 | 0.70 |
| LOXL1 (203570_at) | 0.52 | 0.54 | −0.35 | 0.82 |
| LOXL2 (202998_s_at) | 0.22 | 0.33 | −0.04 | 0.70 |
| LPL (203548_s_at) | 0.71 | 0.72 | −0.46 | 0.42 |
| LPL (203549_s_at) | 0.74 | 0.72 | −0.28 | 0.45 |
| LRRC15 (213909_at) | 0.31 | 0.36 | −0.13 | 0.71 |
| LRRC32 (203835_at) | 0.68 | 0.72 | −0.49 | 0.76 |
| LTBP2 (204682_at) | 0.58 | 0.70 | −0.22 | 0.68 |
| LUZP5 (219588_s_at) | −0.30 | −0.38 | 0.72 | −0.27 |
| MAD2L1 (203362_s_at) | −0.40 | −0.48 | 0.83 | −0.32 |
| MCAM (210869_s_at) | 0.68 | 0.66 | −0.38 | 0.52 |
| MCM2 (202107_s_at) | −0.16 | −0.31 | 0.66 | −0.29 |
| MCM4 (222036_s_at) | −0.33 | −0.50 | 0.63 | −0.24 |
| MCM4 (222037_at) | −0.24 | −0.41 | 0.65 | −0.27 |
| MCM7 (208795_s_at) | −0.41 | −0.47 | 0.78 | −0.45 |
| MEF2C (209200_at) | 0.71 | 0.77 | −0.60 | 0.45 |
| MELK (204825_at) | −0.41 | −0.44 | 0.74 | −0.32 |
| MFAP2 (203417_at) | 0.36 | 0.36 | −0.27 | 0.78 |
| MFAP5 (209758_s_at) | 0.37 | 0.33 | −0.03 | 0.64 |
| MFAP5 (213765_at) | 0.45 | 0.43 | −0.08 | 0.64 |
| MFGE8 (210605_s_at) | 0.52 | 0.59 | −0.33 | 0.64 |
| MKI67 (212021_s_at) | −0.38 | −0.42 | 0.69 | −0.36 |
| MKI67 (212022_s_at) | −0.36 | −0.51 | 0.63 | −0.13 |
| MLF1IP (218883_s_at) | −0.40 | −0.48 | 0.84 | −0.36 |
| MMP14 (202828_s_at) | 0.24 | 0.25 | −0.01 | 0.63 |
| MMP2 (201069_at) | 0.43 | 0.55 | −0.31 | 0.78 |
| MMRN2 (219091_s_at) | 0.53 | 0.65 | −0.41 | 0.21 |
| MXRA7 (212509_s_at) | 0.72 | 0.76 | −0.31 | 0.69 |
| MYBL2 (201710_at) | −0.29 | −0.35 | 0.83 | −0.14 |
| MYH11 (201497_x_at) | 0.59 | 0.69 | −0.59 | 0.42 |
| MYL9 (201058_s_at) | 0.56 | 0.61 | −0.24 | 0.76 |
| MYLK (202555_s_at) | 0.70 | 0.76 | −0.38 | 0.48 |
| NBL1 (201621_at) | 0.40 | 0.32 | −0.20 | 0.64 |
| NEK2 (204641_at) | −0.47 | −0.52 | 0.66 | −0.31 |
| NID1 (202007_at) | 0.64 | 0.72 | −0.44 | 0.77 |
| NID1 (202008_s_at) | 0.70 | 0.73 | −0.48 | 0.87 |
| NNMT (202238_s_at) | 0.51 | 0.53 | −0.21 | 0.66 |
| NRN1 (218625_at) | 0.68 | 0.75 | −0.26 | 0.39 |
| NRP1 (212298_at) | 0.55 | 0.69 | −0.33 | 0.40 |
| NUSAP1 (218039_at) | −0.39 | −0.45 | 0.86 | −0.32 |
| NUSAP1 (219978_s_at) | −0.52 | −0.59 | 0.84 | −0.35 |
| OLFML2B (213125_at) | 0.44 | 0.52 | −0.24 | 0.76 |
| OMD (205907_s_at) | 0.48 | 0.48 | −0.41 | 0.63 |
| PBK (219148_at) | −0.31 | −0.42 | 0.73 | −0.28 |
| PCOLCE (202465_at) | 0.55 | 0.57 | −0.22 | 0.81 |
| PDGFRA (203131_at) | 0.73 | 0.75 | −0.39 | 0.79 |
| PDGFRL (205226_at) | 0.69 | 0.69 | −0.56 | 0.78 |
| PDPN (221898_at) | 0.47 | 0.51 | −0.22 | 0.66 |
| PDZK3 (209493_at) | 0.56 | 0.67 | −0.52 | 0.47 |
| PECAM1 (208982_at) | 0.67 | 0.74 | −0.41 | 0.25 |
| PECAM1 (208983_s_at) | 0.66 | 0.63 | −0.39 | 0.29 |
| PLEKHC1 (209209_s_at) | 0.67 | 0.63 | −0.29 | 0.68 |
| PLEKHC1 (209210_s_at) | 0.62 | 0.64 | −0.28 | 0.65 |
| PLK1 (202240_at) | −0.16 | −0.29 | 0.69 | −0.18 |
| PLSCR4 (218901_at) | 0.81 | 0.87 | −0.65 | 0.71 |
| PLXDC1 (219700_at) | 0.61 | 0.66 | −0.36 | 0.66 |
| PPAP2A (209147_s_at) | 0.69 | 0.70 | −0.53 | 0.39 |
| PPAP2B (209355_s_at) | 0.60 | 0.63 | −0.69 | 0.39 |
| PRC1 (218009_s_at) | −0.45 | −0.52 | 0.90 | −0.37 |
| PROS1 (207808_s_at) | 0.85 | 0.90 | −0.60 | 0.55 |
| PRRX1 (205991_s_at) | 0.59 | 0.57 | −0.27 | 0.79 |
| PSF1 (206102_at) | −0.44 | −0.46 | 0.74 | −0.36 |
| PTGDS (211663_x_at) | 0.64 | 0.65 | −0.31 | 0.40 |
| PTGDS (211748_x_at) | 0.66 | 0.65 | −0.37 | 0.49 |
| PTGIS (208131_s_at) | 0.71 | 0.68 | −0.32 | 0.54 |
| PTN (211737_x_at) | 0.52 | 0.65 | −0.59 | 0.49 |
| PTTG1 (203554_x_at) | −0.44 | −0.51 | 0.85 | −0.39 |
| RAB23 (220955_x_at) | 0.40 | 0.37 | −0.21 | 0.76 |
| RACGAP1 (222077_s_at) | −0.37 | −0.45 | 0.83 | −0.28 |
| RARRES2 (209496_at) | 0.51 | 0.56 | −0.09 | 0.71 |
| RBMS1 (207266_x_at) | 0.70 | 0.67 | −0.44 | 0.52 |
| RCN3 (61734_at) | 0.16 | 0.22 | −0.07 | 0.67 |
| RECK (205407_at) | 0.64 | 0.73 | −0.58 | 0.69 |
| RFC4 (204023_at) | −0.34 | −0.39 | 0.67 | −0.22 |
| RGC32 (218723_s_at) | 0.66 | 0.64 | −0.39 | 0.60 |
| RNASEH2A (203022_at) | −0.44 | −0.48 | 0.79 | −0.33 |
| RRM2 (201890_at) | −0.28 | −0.40 | 0.82 | −0.19 |
| RRM2 (209773_s_at) | −0.41 | −0.53 | 0.87 | −0.26 |
| SAA1 (214456_x_at) | 0.65 | 0.74 | −0.44 | 0.43 |
| SDC2 (212158_at) | 0.23 | 0.23 | −0.04 | 0.65 |
| SEMA3G (219689_at) | 0.70 | 0.73 | −0.49 | 0.29 |
| SERPINF1 (202283_at) | 0.66 | 0.63 | −0.19 | 0.69 |
| SFRP4 (204051_s_at) | 0.75 | 0.69 | −0.54 | 0.67 |
| SH3BP5 (201811_x_at) | 0.70 | 0.64 | −0.45 | 0.46 |
| SIL (205339_at) | −0.39 | −0.43 | 0.69 | −0.39 |
| SLIT2 (209897_s_at) | 0.72 | 0.73 | −0.52 | 0.53 |
| SLIT3 (203813_s_at) | 0.69 | 0.70 | −0.56 | 0.64 |
| SMC2L1 (204240_s_at) | −0.32 | −0.37 | 0.71 | −0.25 |
| SMC4L1 (201663_s_at) | −0.25 | −0.34 | 0.63 | −0.31 |
| SNAI2 (213139_at) | 0.61 | 0.67 | −0.40 | 0.80 |
| SPAG5 (203145_at) | −0.46 | −0.53 | 0.81 | −0.40 |
| SPARC (212667_at) | 0.62 | 0.69 | −0.50 | 0.86 |
| SPARCL1 (200795_at) | 0.74 | 0.82 | −0.64 | 0.66 |
| SPHK1 (219257_s_at) | 0.25 | 0.27 | 0.03 | 0.68 |
| SPON1 (209436_at) | 0.58 | 0.65 | −0.50 | 0.82 |
| SPON1 (209437_s_at) | 0.52 | 0.49 | −0.30 | 0.76 |
| SPON2 (218638_s_at) | 0.45 | 0.57 | −0.31 | 0.75 |
| SRPX (204955_at) | 0.76 | 0.80 | −0.28 | 0.62 |
| ST5 (202440_s_at) | 0.39 | 0.55 | −0.36 | 0.65 |
| STEAP1 (205542_at) | 0.29 | 0.23 | −0.63 | 0.28 |
| STK6 (204092_s_at) | −0.35 | −0.42 | 0.80 | −0.28 |
| STK6 (208079_s_at) | −0.38 | −0.42 | 0.80 | −0.30 |
| SVEP1 (213247_at) | 0.66 | 0.75 | −0.51 | 0.58 |
| TACC3 (218308_at) | −0.18 | −0.30 | 0.75 | −0.21 |
| TAGLN (205547_s_at) | 0.52 | 0.60 | −0.27 | 0.78 |
| TCF4 (212382_at) | 0.62 | 0.69 | −0.59 | 0.61 |
| THBD (203887_s_at) | 0.63 | 0.56 | −0.32 | 0.34 |
| THBS2 (203083_at) | 0.47 | 0.55 | −0.24 | 0.77 |
| THEM2 (204565_at) | −0.24 | −0.23 | 0.66 | −0.24 |
| THY1 (208850_s_at) | 0.44 | 0.48 | −0.34 | 0.82 |
| TNFAIP6 (206025_s_at) | 0.30 | 0.34 | −0.25 | 0.69 |
| TNS1 (221748_s_at) | 0.82 | 0.88 | −0.48 | 0.60 |
| TOP2A (201291_s_at) | −0.33 | −0.43 | 0.79 | −0.30 |
| TOP2A (201292_at) | −0.37 | −0.44 | 0.81 | −0.33 |
| TPM2 (204083_s_at) | 0.47 | 0.54 | −0.11 | 0.65 |
| TPX2 (210052_s_at) | −0.34 | −0.44 | 0.88 | −0.26 |
| TRIP13 (204033_at) | −0.47 | −0.56 | 0.85 | −0.41 |
| TROAP (204649_at) | −0.33 | −0.43 | 0.87 | −0.32 |
| TYMS (202589_at) | −0.17 | −0.23 | 0.67 | −0.14 |
| UBE2C (202954_at) | −0.52 | −0.61 | 1.00 | −0.41 |
| UBE2S (202779_s_at) | −0.55 | −0.62 | 0.69 | −0.39 |
| WISP1 (206796_at) | 0.47 | 0.48 | −0.48 | 0.74 |
| WWTR1 (202132_at) | 0.65 | 0.67 | −0.45 | 0.60 |
| WWTR1 (202133_at) | 0.62 | 0.72 | −0.53 | 0.64 |
| ZBTB20 (205383_s_at) | 0.46 | 0.61 | −0.65 | 0.33 |
| ZNF423 (214761_at) | 0.68 | 0.73 | −0.56 | 0.71 |
| ZWINT (204026_s_at) | −0.41 | −0.50 | 0.75 | −0.36 |

For the separation of the aggregate breast cancer tumor class CD into elementary breast cancer response classes C and D, one of the following partial classifiers is used:
1. This majority voting scheme consists of the measurement of genes IGF1 (Affymetrix Probeset IDs 209540_at, 209541_at, or 209542_x_at), FHL1 (Affymetrix Probeset IDs 201640_at or 214505_s_at), EFEMP1 (201842_s_at), IL6ST (212195_at), SPARCL1 (200795_at), NET1 (201830_s_at), ISLR (207191_s_at), ENO1 (217294_s_at), and CDH5 (204677_at). Any of the following conditions is evaluated and counts as a vote for C if fulfilled:
   a) Expression of IGF1 (209540_at)>155
   b) Expression of IGF1 (209541_at)>321
   c) Expression of IGF1 (209542_x_at)>184
   d) Expression of FHL1 (201540_at)>847
   e) Expression of FHL1 (214505_s_at)>281
   f) Expression of EFEMP1 (201842)>1759
   g) Expression of IL6ST (212915_at)>1347
   h) Expression of SPARCL1 (200795_at)>2518
   i) Expression of NET1 (201830_s_at)>2434
   j) Expression of ISLR (207191_s_a)>1007
   k) Expression of ENO1 (217297_s_at)<3476
   l) Expression of CDH5 (204677_at)>240
   If there is a total of than 5.5 votes for breast cancer response group C, the unknown tissue sample is predicted as breast cancer response group C; otherwise, it is predicted a breast cancer response group D.
2. An alternative voting scheme uses the genes IGF1 and FHL1 only (see above for Affymetrix Probeset IDs). If there are more than two votes for group C, the unknown sample is predicted to be in group C; if not, it is predicted to be in group D.
3. An alternative voting scheme uses any combination of a single probeset for IGF1 and a single probeset for FHL1. If there is more than one vote for group C, the unknown sample is predicted to be in group C; if not, it is predicted to be in group D.
4. An alternative predictor uses just one the genes listed in 1. and predicts the breast cancer response class according to its vote.

### Example 3

### Significance of Correlated Marker Genes

It is well known that expression level data of multiple genes can be highly redundant information, due to co-regulation of certain genes or groups of genes in living organisms.

According to the invention, a so-called “correlation coefficient” is used as a measure for the degree of similarity of expression levels in multiple samples which corresponds to the degree of similarity of the information contained in these genes. If we denote the log expression value of the i-th gene (i=1, 2, 3, . . . N) of patient j (i=1, 2, 3, . . . M) by g_{i j}, the correlation coefficient r may be defined as

$${r}_{i 1 , i 2} := \frac{{\sum }_{j = 1}^{M} ( {g}_{i 1 , j} - {\overline{g}}_{i 1} ) \cdot ( {g}_{i 2 , j} - {\overline{g}}_{i 2} )}{\sqrt{( {\sum }_{j = 1}^{M} {( {g}_{i 1 , j} - {\overline{g}}_{i 1} )}^{2} ) \cdot ( {\sum }_{j = 1}^{M} {( {g}_{i 2 , j} - {\overline{g}}_{i 2} )}^{2} )}}$$

where the mean value of gene i is given by

$${\overline{g}}_{i} := \frac{1}{M} {\sum }_{j = 1}^{M} {g}_{i , j} .$$

r is also called “Pearson Correlation Coefficient” and is widely used in the statistical community.

While r may take any value between (and including) −1 and 1, correlations with an absolute value close to 1 indicate a linear relationship between the genes under consideration, meaning that the two genes carry virtually the same information.

In the context of the present invention it is apparent that genes sharing a sufficiently large correlation coefficient with marker genes of the preceding examples can equally well be used in the classification method, because they provide almost identical information.

Tables 1-5b list genes with a high correlation to marker genes used in the Examples. They can be used in the separation of breast cancer response classes AB and CD from aggregate class ABCD (Table 1 and/or 2), and for the separation of breast cancer response classes A and B from aggregate class AB (Table 3 and/or 4), and finally for the separation of breast cancer response classes C and D from aggregate class CD (Table 5a and 5b).

A “sufficiently large correlation coefficient”, in this context, needs to be explained in more detail. To keep the gene lists fair and short, we identified genes that had an unusually high correlation with a probability of p<0.05 already including a conservative Bonferroni correction (that is, p has to be divided by the number of genes checked for high correlation, in this case, N=22284 for Affymetrix HG U133A chip used here) which yields an effective p value of P_{eff}<0.05/22284=2.24e−6. However, this approach is overly conservative in the case of expression data since many if not most genes measured on the chip have little if no contributing information to the prediction at hand. Based on all samples, filtering was carried out in an unsupervised manner, that is, without using knowledge about clinical follow-up or class membership. This preprocessing step is frequently practiced as presented or in variations, widely used and known to any person skilled in the art.

In the invention at hand, genes were tested for 1) measurability, and 2) for information content. Technical measurability was assessed by only considering genes that were “present” in at least 5% of the samples. “Present” (or “Present Calls”) is an information given by Affymetrix' MASS software for each sample and each gene. It is part of Affymetrix' standard protocol and available to persons skilled in the art. Measurability in terms of a signal-to-noise ratio was assessed by estimating a technical noise and considering only genes that (in median) had a signal intensity above a given noise level. Here, only genes with a median expression of at least 200 were considered. Lastly, information content was measured by estimation variations in genes across all samples by computing the coefficient of variance (CV). In this invention, all genes considered further had a coefficient of variance of at least 50%, which is an arbitrarily chosen value based on experience.

The use of these three filters (Present Calls, median expression, CV) yields a much shorter list of considerable genes. Of the roughly 22000 genes on the chip, 2726 fulfilled the unsupervised criteria. So, for the p value under consideration, a Bonferroni correction with N=2726 can be used instead of N=22284, which gives an effective p value of P_{eff}<0.05/2726=1.83e−5. Using a (two-sided) Student's t statistic, we can compute the minimum correlation coefficient rₘᵢₙ from P_{eff}, also taking the sample number at each separation point into account. Finally, the following minimal correlation values and numbers of correlated genes were obtained:

| | Number of samples | | Resulting number |
|---|---|---|---|
| Separation | in finding cohort | rₘᵢₙ | of correlated genes |
| AB <-> CD | 50 | 0.5489 | 427 |
| A <-> B | 37 | 0.6241 | 339 |
| C <-> D | 13 | 0.8946 | 35 |

Thus, genes having a correlation coefficient equal to or larger than rₘᵢₙ to the marker genes of Example 2 of the present invention, are further preferred marker genes for the separation of AB and CD, A and B, and C and D in a classification tree of the invention.

Further preferred marker genes are genes whose gene expression is correlated with the one of marker genes of Example 2 with a correlation coefficient in one of Tables 1, 2, 3, 4, 5a or 5b of preferably 0.6, 0.7, 0.8, 0.9, 0.95, 0.99, 0.999 or most preferably 1.

Also preferred marker genes are genes whose gene expression is correlated with at least one marker gene of Example 2 with a correlation coefficient of preferably 0.7, 0.8, 0.9, 0.95, 0.99 or most preferably 1 in a separate series of expression level measurements.

Further preferred marker genes are genes whose gene expression is previously known to be highly correlated with one of marker genes of Example 2.

### Example 4

### Advantage of a Majority Voting Scheme Over Univariate Classification in Certain Cases

Univariate classification, in its simplest embodiment, compares a gene expression value with a pre-determined threshold value. If the expression value is smaller than the threshold value, the sample is predicted to belong to the first elementary class (or aggregate class), and the second elementary class (or aggregate class) if it is not. If the decision for a treatment is based on this simple predictor, the quality of the predictor relies solely on the measurement quality of this single gene expression. It is not a robust predictor in terms of reliability.

If one takes measurements of expression values of a number of genes (typically, this number is small) and uses each gene as a univariate classifier, but then uses the combined information to predict a class, the information is a lot more reliable and robust. Robustness here means that if a gene cannot be measured correctly for some reason, its influence on the final prediction shall be comparatively small. In the case of a genuine majority vote, it is unlikely that the predictor will switch if just one of its univariate partial predictors (called “votes”) does.

It is obviously an advantage to use predictors that are more robust in order to obtain more reliable results from a technical point of view. It is even more obvious in view of the application, that is, predicting responders and non-responders for a taxane-based therapy where it is ethically necessary to allow a patient the optimal treatment. Misidentifying a responder as a non-responder to this therapy will possibly deny this patient a successful therapeutic strategy which has to be avoided.

TABLE 5a
Separation of Taxane Response Classes C vs. D. Genes with SIGNIFICANT correlation
coefficients (|cc| > 0.89)

| | IGF1 | IGF1 | IGF1 | FHL1 | FHL1 |
|---|---|---|---|---|---|
| | (209540_at) | (209541_at) | (209542_x_at) | (201539_s_at) | (201540_at) |
| CCL14 /// CCL15 (205392_s_at) | 0.76 | 0.83 | 0.94 | 0.56 | 0.59 |
| CDH5 (204677_at) | 0.73 | 0.84 | 0.85 | 0.64 | 0.82 |
| DAB2 (201279_s_at) | 0.82 | 0.75 | 0.68 | 0.63 | 0.90 |
| DAB2 (201280_s_at) | 0.82 | 0.70 | 0.67 | 0.54 | 0.90 |
| DDX17 (213998_s_at) | −0.30 | −0.33 | −0.31 | −0.25 | −0.65 |
| EFEMP1 (201842_s_at) | 0.29 | 0.36 | 0.42 | 0.52 | 0.71 |
| EFEMP1 (201843_s_at) | 0.32 | 0.41 | 0.44 | 0.52 | 0.63 |
| ENO1 (217294_s_at) | −0.89 | −0.79 | −0.80 | −0.28 | −0.56 |
| F13A1 (203305_at) | 0.72 | 0.61 | 0.53 | 0.66 | 0.91 |
| FHL1 (201539_s_at) | 0.41 | 0.52 | 0.58 | 1.00 | 0.70 |
| FHL1 (201540_at) | 0.77 | 0.71 | 0.69 | 0.70 | 1.00 |
| FHL1 (210298_x_at) | 0.57 | 0.57 | 0.56 | 0.79 | 0.93 |
| FHL1 (214505_s_at) | 0.63 | 0.55 | 0.59 | 0.64 | 0.95 |
| GPR116 (212950_at) | 0.66 | 0.80 | 0.74 | 0.76 | 0.80 |
| IGF1 (209540_at) | 1.00 | 0.85 | 0.79 | 0.41 | 0.77 |
| IGF1 (209541_at) | 0.85 | 1.00 | 0.90 | 0.52 | 0.71 |
| IGF1 (209542_x_at) | 0.79 | 0.90 | 1.00 | 0.58 | 0.69 |
| IGFBP4 (201508_at) | 0.91 | 0.83 | 0.72 | 0.54 | 0.82 |
| IL6ST (204863_s_at) | 0.74 | 0.52 | 0.48 | 0.24 | 0.35 |
| IL6ST (204864_s_at) | 0.35 | 0.50 | 0.26 | 0.08 | 0.04 |
| IL6ST (211000_s_at) | 0.29 | 0.07 | 0.12 | −0.07 | 0.06 |
| IL6ST (212195_at) | 0.79 | 0.62 | 0.49 | 0.27 | 0.58 |
| IL6ST (212196_at) | 0.65 | 0.66 | 0.43 | 0.36 | 0.57 |
| ISLR (207191_s_at) | 0.30 | 0.28 | 0.28 | 0.33 | 0.51 |
| MAN1C1 (218918_at) | 0.92 | 0.76 | 0.74 | 0.26 | 0.73 |
| MMRN2 (219091_s_at) | 0.77 | 0.87 | 0.92 | 0.70 | 0.69 |
| MUC1 (207847_s_at) | −0.39 | −0.54 | −0.48 | −0.08 | −0.39 |
| NET1 (201830_s_at) | 0.43 | 0.53 | 0.52 | 0.21 | 0.56 |
| PLVAP (221529_s_at) | 0.52 | 0.75 | 0.75 | 0.71 | 0.69 |
| SPARCL1 (200795_at) | 0.93 | 0.94 | 0.92 | 0.49 | 0.74 |
| SVEP1 (213247_at) | 0.69 | 0.74 | 0.61 | 0.68 | 0.92 |
| TBRG4 (220789_s_at) | −0.82 | −0.85 | −0.84 | −0.50 | −0.60 |
| THBS4 (204776_at) | 0.80 | 0.84 | 0.79 | 0.44 | 0.62 |
| WEE1 (215711_s_at) | −0.91 | −0.71 | −0.71 | −0.13 | −0.59 |
| WISP2 (205792_at) | 0.88 | 0.81 | 0.83 | 0.43 | 0.64 |

| | FHL1 | FHL1 | EFEMP1 | EFEMP1 |
|---|---|---|---|---|
| | (210298_x_at) | (214505_s_at) | (201842_s_at) | (201843_s_at) |
| CCL14 /// CCL15 (205392_s_at) | 0.48 | 0.48 | 0.31 | 0.39 |
| CDH5 (204677_at) | 0.75 | 0.75 | 0.62 | 0.62 |
| DAB2 (201279_s_at) | 0.79 | 0.84 | 0.55 | 0.53 |
| DAB2 (201280_s_at) | 0.82 | 0.89 | 0.66 | 0.60 |
| DDX17 (213998_s_at) | −0.71 | −0.79 | −0.90 | −0.79 |
| EFEMP1 (201842_s_at) | 0.85 | 0.84 | 1.00 | 0.94 |
| EFEMP1 (201843_s_at) | 0.77 | 0.73 | 0.94 | 1.00 |
| ENO1 (217294_s_at) | −0.39 | −0.44 | −0.24 | −0.36 |
| F13A1 (203305_at) | 0.80 | 0.82 | 0.43 | 0.31 |
| FHL1 (201539_s_at) | 0.79 | 0.64 | 0.52 | 0.52 |
| FHL1 (201540_at) | 0.93 | 0.95 | 0.71 | 0.63 |
| FHL1 (210298_x_at) | 1.00 | 0.94 | 0.85 | 0.77 |
| FHL1 (214505_s_at) | 0.94 | 1.00 | 0.84 | 0.73 |
| GPR116 (212950_at) | 0.77 | 0.70 | 0.55 | 0.54 |
| IGF1 (209540_at) | 0.57 | 0.63 | 0.29 | 0.32 |
| IGF1 (209541_at) | 0.57 | 0.55 | 0.36 | 0.41 |
| IGF1 (209542_x_at) | 0.56 | 0.59 | 0.42 | 0.44 |
| IGFBP4 (201508_at) | 0.71 | 0.72 | 0.47 | 0.53 |
| IL6ST (204863_s_at) | 0.16 | 0.16 | −0.20 | −0.09 |
| IL6ST (204864_s_at) | −0.10 | −0.23 | −0.36 | −0.19 |
| IL6ST (211000_s_at) | −0.10 | −0.06 | −0.26 | −0.19 |
| IL6ST (212195_at) | 0.48 | 0.42 | 0.20 | 0.26 |
| IL6ST (212196_at) | 0.53 | 0.38 | 0.26 | 0.30 |
| ISLR (207191_s_at) | 0.42 | 0.42 | 0.31 | 0.29 |
| MAN1C1 (218918_at) | 0.53 | 0.65 | 0.33 | 0.29 |
| MMRN2 (219091_s_at) | 0.63 | 0.58 | 0.46 | 0.56 |
| MUC1 (207847_s_at) | −0.33 | −0.29 | −0.37 | −0.38 |
| NET1 (201830_s_at) | 0.47 | 0.47 | 0.53 | 0.52 |
| PLVAP (221529_s_at) | 0.69 | 0.63 | 0.62 | 0.64 |
| SPARCL1 (200795_at) | 0.58 | 0.63 | 0.37 | 0.42 |
| SVEP1 (213247_at) | 0.91 | 0.85 | 0.71 | 0.67 |
| TBRG4 (220789_s_at) | −0.45 | −0.48 | −0.24 | −0.34 |
| THBS4 (204776_at) | 0.51 | 0.47 | 0.36 | 0.47 |
| WEE1 (215711_s_at) | −0.35 | −0.50 | −0.20 | −0.22 |
| WISP2 (205792_at) | 0.53 | 0.57 | 0.40 | 0.48 |

TABLE 5b
Separation of Taxane Response Classes C vs. D. Genes with SIGNIFICANT correlation
coefficients (|cc| > 0.89, continued):

| | IL6ST | IL6ST | IL6ST | IL6ST | IL6ST |
|---|---|---|---|---|---|
| | (204863_s_at) | (204864_s_at) | (211000_s_at) | (212195_at) | (212196_at) |
| CCL14 /// CCL15 (205392_s_at) | 0.49 | 0.30 | 0.11 | 0.53 | 0.44 |
| CDH5 (204677_at) | 0.45 | 0.13 | −0.05 | 0.51 | 0.50 |
| DAB2 (201279_s_at) | 0.45 | 0.10 | 0.10 | 0.50 | 0.47 |
| DAB2 (201280_s_at) | 0.40 | −0.09 | −0.03 | 0.57 | 0.48 |
| DDX17 (213998_s_at) | 0.24 | 0.44 | 0.37 | −0.13 | −0.17 |
| EFEMP1 (201842_s_at) | −0.20 | −0.36 | −0.26 | 0.20 | 0.26 |
| EFEMP1 (201843_s_at) | −0.09 | −0.19 | −0.19 | 0.26 | 0.30 |
| ENO1 (217294_s_at) | −0.70 | −0.33 | −0.32 | −0.75 | −0.58 |
| F13A1 (203305_at) | 0.43 | 0.12 | 0.09 | 0.50 | 0.51 |
| FHL1 (201539_s_at) | 0.24 | 0.08 | −0.07 | 0.27 | 0.36 |
| FHL1(201540_at) | 0.35 | 0.04 | 0.06 | 0.58 | 0.57 |
| FHL1 (210298_x_at) | 0.16 | −0.10 | −0.10 | 0.48 | 0.53 |
| FHL1 (214505_s_at) | 0.16 | −0.23 | −0.06 | 0.42 | 0.38 |
| GPR116 (212950_at) | 0.45 | 0.22 | −0.18 | 0.47 | 0.54 |
| IGF1 (209540_at) | 0.74 | 0.35 | 0.29 | 0.79 | 0.65 |
| IGF1 (209541_at) | 0.52 | 0.50 | 0.07 | 0.62 | 0.66 |
| IGF1 (209542_x_at) | 0.48 | 0.26 | 0.12 | 0.49 | 0.43 |
| IGFBP4 (201508_at) | 0.60 | 0.27 | 0.16 | 0.70 | 0.65 |
| IL6ST (204863_s_at) | 1.00 | 0.57 | 0.53 | 0.70 | 0.49 |
| IL6ST (204864_s_at) | 0.57 | 1.00 | 0.43 | 0.49 | 0.58 |
| 1L6ST (211000_s_at) | 0.53 | 0.43 | 1.00 | 0.39 | 0.18 |
| IL6ST (212195_at) | 0.70 | 0.49 | 0.39 | 1.00 | 0.91 |
| IL6ST (212196_at) | 0.49 | 0.58 | 0.18 | 0.91 | 1.00 |
| ISLR (207191_s_at) | 0.14 | 0.12 | 0.45 | 0.28 | 0.28 |
| MAN1C1 (218918_at) | 0.58 | 0.11 | 0.18 | 0.70 | 0.56 |
| MMRN2 (219091_s_at) | 0.58 | 0.34 | 0.16 | 0.57 | 0.53 |
| MUC1 (207847_s_at) | −0.26 | −0.46 | −0.38 | −0.58 | −0.59 |
| NET1 (201830_s_at) | 0.19 | 0.31 | 0.36 | 0.52 | 0.50 |
| PLVAP (221529_s_at) | 0.20 | 0.07 | −0.20 | 0.26 | 0.37 |
| SPARCL1 (200795_at) | 0.59 | 0.32 | 0.17 | 0.66 | 0.60 |
| SVEP1 (213247_at) | 0.24 | 0.21 | 0.05 | 0.61 | 0.71 |
| TBRG4 (220789_s_at) | −0.65 | −0.32 | −0.09 | −0.49 | −0.42 |
| THBS4 (204776_at) | 0.50 | 0.35 | 0.18 | 0.71 | 0.66 |
| WEE1 (215711_s_at) | −0.60 | −0.19 | −0.30 | −0.66 | −0.43 |
| WISP2 (205792_at) | 0.54 | 0.19 | 0.21 | 0.63 | 0.48 |

| | SPARCL1 | NET1 | ISLR | ENO1 | CDH5 |
|---|---|---|---|---|---|
| | (200795_at) | (201830_s_at) | (207191_s_at) | (217294_s_at) | (204677_at) |
| CCL14 /// CCL15 (205392_s_at) | 0.85 | 0.48 | 0.21 | −0.81 | 0.72 |
| CDH5 (204677_at) | 0.83 | 0.54 | 0.38 | −0.73 | 1.00 |
| DAB2 (201279_s_at) | 0.82 | 0.33 | 0.48 | −0.63 | 0.80 |
| DAB2 (201280_s_at) | 0.79 | 0.38 | 0.34 | −0.68 | 0.83 |
| DDX17 (213998_s_at) | −0.36 | −0.40 | −0.23 | 0.23 | −0.59 |
| EFEMP1 (201842_s_at) | 0.37 | 0.53 | 0.31 | −0.24 | 0.62 |
| EFEMP1 (201843_s_at) | 0.42 | 0.52 | 0.29 | −0.36 | 0.62 |
| ENO1 (217294_s_at) | −0.89 | −0.50 | −0.35 | 1.00 | −0.73 |
| F13A1 (203305_at) | 0.65 | 0.28 | 0.44 | −0.39 | 0.65 |
| FHL1 (201539_s_at) | 0.49 | 0.21 | 0.33 | −0.28 | 0.64 |
| FHL1(201540_at) | 0.74 | 0.56 | 0.51 | −0.56 | 0.82 |
| FHL1 (210298_x_at) | 0.58 | 0.47 | 0.42 | −0.39 | 0.75 |
| FHL1 (214505_s_at) | 0.63 | 0.47 | 0.42 | −0.44 | 0.75 |
| GPR116 (212950_at) | 0.73 | 0.40 | 0.28 | −0.57 | 0.94 |
| IGF1 (209540_at) | 0.93 | 0.43 | 0.30 | −0.89 | 0.73 |
| IGF1 (209541_at) | 0.94 | 0.53 | 0.28 | −0.79 | 0.84 |
| IGF1 (209542_x_at) | 0.92 | 0.52 | 0.28 | −0.80 | 0.85 |
| IGFBP4 (201508_at) | 0.91 | 0.37 | 0.42 | −0.81 | 0.81 |
| IL6ST (204863_s_at) | 0.59 | 0.19 | 0.14 | −0.70 | 0.45 |
| IL6ST (204864_s_at) | 0.32 | 0.31 | 0.12 | −0.33 | 0.13 |
| 1L6ST (211000_s_at) | 0.17 | 0.36 | 0.45 | −0.32 | −0.05 |
| IL6ST (212195_at) | 0.66 | 0.52 | 0.28 | −0.75 | 0.51 |
| IL6ST (212196_at) | 0.60 | 0.50 | 0.28 | −0.58 | 0.50 |
| ISLR (207191_s_at) | 0.34 | 0.54 | 1.00 | −0.35 | 0.38 |
| MAN1C1 (218918_at) | 0.90 | 0.37 | 0.32 | −0.85 | 0.73 |
| MMRN2 (219091_s_at) | 0.88 | 0.48 | 0.26 | −0.79 | 0.88 |
| MUC1 (207847_s_at) | −0.42 | −0.91 | −0.29 | 0.47 | −0.48 |
| NET1 (201830_s_at) | 0.44 | 1.00 | 0.54 | −0.50 | 0.54 |
| PLVAP (221529_s_at) | 0.72 | 0.38 | 0.46 | −0.57 | 0.90 |
| SPARCL1 (200795_at) | 1.00 | 0.44 | 0.34 | −0.89 | 0.83 |
| SVEP1 (213247_at) | 0.72 | 0.57 | 0.52 | −0.50 | 0.74 |
| TBRG4 (220789_s_at) | −0.91 | −0.23 | −0.32 | 0.83 | −0.83 |
| THBS4 (204776_at) | 0.87 | 0.54 | 0.47 | −0.92 | 0.75 |
| WEE1 (215711_s_at) | −0.84 | −0.44 | −0.30 | 0.90 | −0.59 |
| WISP2 (205792_at) | 0.89 | 0.42 | 0.21 | −0.90 | 0.72 |

### LITERATURE

(1) Chang J C, Wooten E C, Tsimelzon A, Hilsenbeck S G, Gutierrez M C, Elledge R, Mohsin S, Osborne C K, Chamness G C, Allred D C, O'Connell P. Gene expression profiling for the prediction of therapeutic response to docetaxel in patients with breast cancer. Lancet, 362:362-369, 2003.
(2) Goldhirsch A, Wood W C, Gelber R D, Coates A S, Thulimann B, Senn H J. Meeting Highlights: updated international expert consensus on the primary therapy of early breast cancer. J Clin Oncol 21: 3357-3365, 2003
(3) Early Breast Cancer Trialists' Collaborative Group. Polychemotherapy for early breast cancer: an overview of the randomised trials. Lancet 352: 930-942, 1998
(4) Early Breast Cancer Trialists' Collaborative Group. Tamoxifen for early breast cancer: an overview of the randomised trials. Lancet 351: 1451-1467, 1998
(5) Ganz P A, Desmond K A, Leedham B, Rowland J H, Meyerowitz B E, Belin T R. Quality of life in long-term, disease-free survivors of breast cancer: a follow-up study. J Natl Cancer Inst 94: 39-49, 2002
(6) Ayers M, Symmans W F, Stec J, Damokosh A I, Clark E, Hess K, Lecocke M, Metivier J, Booser D, Ibrahim N, Valero V, Royce M, Arun B, Whitman G, Ross J, Sneige N, Hortobagyi G N, Pusztai L. Gene expression profiles predict complete pathologic response to neoadjuvant paclitaxel and fluorouracil, doxorubicin, and cyclophosphamide chemotherapy in breast cancer. J Clin Oncol 22(12): 2284-2293, 2004
(7) Tong W, Hong H, Fang H, Xie Q, Perkins R. Decision Forest: Combining the Predictions of Multiple Independent Decision Tree Models. J. Chem. Inf. Comput. Sci. 43: 525-531, 2003.
(8) Tong, W, Xie Q, Hong H, Fang H, Shi L, Perkins R, Petricoin E F. Using Decision Forest to Classify Prostate Cancer Samples on the Basis of SELDI-TOF MS Data: Assessing Chance Correlation and Prediction Confidence. Environmental Health Perspectives 112 (16), 2004
(9) Xie Q, Ratnasinghe L D, Hong H, Perkins R, Tang Z Z, Hu N. Taylor P R, Tong W. Decision Forest Analysis of 61 Single Nucleotide Polymorphisms in a Case-Control Study of Esophageal Cancer: A Novel Method. BMC Bioinformatics 6: S4, 2005.

1. Method for the prediction of the response of a breast cancer in a patient to a taxane-based chemotherapy, from a tumour sample of said patient, comprising steps of
(a) determining the expression level of a group of marker genes consisting of (i) a first marker gene selected from the group consisting of ESR1 and WARS and genes co-regulated thereto; and
(ii) a second marker gene selected from the group consisting of CAV1, COL6A2 and UBE2C and genes co-regulated thereto; and
(iii) a third marker gene selected from the group consisting of IGF1, FHL1, EFEMP1, IL6ST, SPARCL1, NET1, ISLR, ENO1 and CDH5 and genes co-regulated thereto;
(b) classifying said sample as belonging to one of several breast cancer response classes from said expression levels of said marker genes, wherein the outcome of said classification is dependent on the expression level of said first marker gene and on the expression level of at least one of said second or said third marker genes;
(c) predicting the response of said breast cancer in said patient to chemotherapy from previously known characteristic properties of tumours of said one of several breast cancer response classes.

2. Method of claim 1, wherein said first marker gene has a correlation coefficient with ESR1 or WARS of equal to or higher than 0.8 in Table 1, or equal to or higher than 0.55 in Table 2; said second marker gene has a correlation coefficient with CAV1, COL6A2 or UBE2C of equal to or higher than 0.8 in Table 3, or equal to or higher than 0.62 in Table 4; and said third marker gene has a correlation with IGF1, FHL1, EFEMP1, IL6ST, SPARCL1, NET1, ISLR, ENO1 or CDH5 of equal to or higher than 0.89 in Table 5a or Table 5b.

3. Method of claim 1, wherein said first marker gene is s ESR1 or WARS; said second marker gene is CAV1, COL6A2 or UBE2C, and said third marker gene is IGF1, FHL1, EFEMP1, IL6ST, SPARCL1, NET1, ISLR, ENO1 or CDH5.

4. Method of claim 1, wherein said several breast cancer response classes are four breast cancer response classes.

5. Method of claim 1, wherein said taxane based chemotherapy is a taxane/anthracycline/cyclophosphamide-based chemotherapy or a Taxotere/Adriamycin/cyclophosphamide-based chemotherapy.

6. Method of claim 1, wherein said determining of the expression level is in a sample taken before the onset of chemotherapy.

7. Method of claim 1, wherein said classification is based on a classification tree.

8. Method of claim 1, wherein said classification involves at least two binary classification steps.

9. Method of claim 1, wherein said classification involves at least one majority voting classification step.

10. Method of claim 1, wherein said classification step (b) is based on a mathematical discriminant function.

11. Method of claim 1, wherein said classification uses a k-nearest-neighbour (kNN) algorithm.

12. Method of claim 1, wherein the chemotherapy is a neoadjuvant chemotherapy.

13. Method of claim 1, wherein the response to chemotherapy is clinical response or pathological response.

14. Method of claim 1, wherein said patient is a human patient.

15. Method of claim 1, wherein said sample of a tumour is a fixed sample, a paraffin-embedded sample, a fresh sample, a fresh frozen sample or a frozen sample.

16. Method of claim 1, wherein said sample of a tumour is from fine needle biopsy, core biopsy or fine needle aspiration.

17. Method of claim 1, wherein said determination of the expression level is by microarray experiment, by RT-PCR, by SAGE, by immunohistochemistry or by TaqMan.

18. A system for predicting the response of a breast cancer in a patient to chemotherapy, comprising
(a) means for determining the expression level of a group of marker genes consisting of
(i) a first marker gene selected from Table 1 or 2; and
(ii) a second marker gene selected from Table 3 or 4; and
(iii) a third marker gene selected from Table 5a or 5b;
(b) classification means, for automatically classifying said sample as belonging to one of several breast cancer response classes from said expression levels of said marker genes, wherein the outcome of said classification is dependent on the expression level of said first marker gene and on the expression level of at least one of said second or said third marker genes;
(c) prediction means for predicting the response of said breast cancer in said patient to chemotherapy from previously known characteristic properties of tumours of said one of several breast cancer response classes.

19. A system of claim 18, wherein said first marker gene is s ESR1 or WARS; said second marker gene is CAV1, COL6A2 or UBE2C, and said third marker gene is IGF1, FHL1, EFEMP1, IL6ST, SPARCL1, NET1, ISLR, ENO1 or CDH5.

20. A system of claim 18, wherein said several breast cancer response classes are four breast cancer response classes.

21. System of claim 18, wherein said means for determining the expression level of a group of marker genes comprises a microarray, a system for 2D gel electrophoresis, a SAGE system or a system for immunohistochemical determination of expression levels.

22. (canceled)
